(19) United States
(12) Patent Application Publication
(10) Pub. No.: US 2020/0069863 A1
(43) Pub. Date: Mar. 5, 2020

(54) STORAGE DEVICE

(71) Applicant: CONCEPTOMED AS, Ballstad (NO)

(72) Inventors: Christian Mide, Ballstad (NO); Marius Andresen, Oslo (NO); Kevin Geers, Oslo (NO); Joachim Wallem Rasmussen, Oslo (NO); Jimmy Gidö Schön, Ballstad (NO); Ken R. Rosendal, Ballstad (NO)

(21) Appl. No.: 16/466,601

(22) PCT Filed: Dec. 5, 2017
(86) PCT No.: PCT/EP2017/081591

§ 371 Date: Jun. 4, 2019

(30) Foreign Application Priority Data
Dec. 5, 2016 (GB) 1620671.6

(51) Int. Cl. A61M 5/00; B65D 83/04; B65D 85/24

(52) U.S. Cl. CPC A61M 5/002; A61M 5/008; B65D 2583/0486; B65D 85/24; B65D 83/0454

(57) ABSTRACT

A storage device (2) for storing a plurality of objects. The storage device (2) comprises: a carousel (8) defining a plurality of cavities (34); a sealing strip (6) extending around the carousel (8) so as to seal the plurality of cavities (34) and a concentric guide structure (18) extending at least partially around the carousel (8). The storage device (2) further comprises a driving member (42) attached to the sealing strip (6). The driving member (42) is arranged such that when moved relative to the guide structure (18), the driving member (42) pulls on the sealing strip (6) and thereby releases a portion of the sealing strip (6) and subsequently wraps the released portion of the sealing strip (6) around the guide structure (18).

The present invention is directed towards a storage device for storing a plurality of objects, for example pen needles.

Many people across the world suffer from diabetes. Whilst some diabetics are able to control their blood sugar levels by adapting their diet and activity levels, many diabetics have to regularly administer insulin injections. Typically, diabetics monitor their blood sugar levels throughout the day and self-administer doses of insulin when necessary to ensure that their blood sugar levels remain at an appropriate level. If blood sugar levels are not maintained at an appropriate level there can be serious harmful effects.

Diabetics often self-administer insulin doses using an insulin pen. The insulin pen typically comprises a cartridge that contains the insulin solution. A pen needle can be removably attached to the insulin pen so as to be in fluid communication with the cartridge. A cartridge will often contain multiple doses of insulin and therefore an insulin pen may be used to administer multiple doses of insulin. The pen needle, however, is intended for single use only, and hence before each use, a new pen needle should be attached to the pen.

Pen needles are typically packaged individually in small, sterile, plastic containers which have a peelable seal which can be removed to gain access to the pen needle. In addition to being contained in a sterile package, the needle tip on the pen needle also often has a separate cover. When a user wishes to employ a new needle in order to administer an insulin dose, they must first remove the seal from the packaging and attach the pen needle to the pen. This may be achieved, for example, by a click fit or by screwing. A user must first remove the outer and inner protective caps from the needle tip before administering a dose. After administering the dose, a user should dispose of the used pen needle along with its packaging and separate cover. Most used needles are disposed in normal household waste and thus potentially represent a contamination risk for third parties.

The insulin pen should be stored without a pen needle attached so that a new, sterile needle can be attached just before it is needed.

A diabetic who suffers from Type I diabetes may need to inject themselves with insulin as many as 5-7 times a day. A diabetic who suffers from Type II diabetes may need to inject themselves between 1-2 times a day. It is therefore apparent that irrespective of the type of diabetes that a person suffers from, it is likely that they will have to inject themselves a number of times each day, and when this is extrapolated over the course of a week or a year it becomes apparent that they will go through a large number of pen needles. Due to the fact that a diabetic may have to administer a dose of insulin throughout the course of the day, ideally a user will carry their insulin pen on their person so that they are able to administer an insulin dose as is necessary. In order to be able to do this, ideally they must also carry a number of sterile pen needles such that they can replace the pen needle with a sterile needle each time they administer a dose. Type I diabetics would therefore ideally carry around seven sterile pen needles on their person.

The requirement for a diabetic to carry around a large number of individually packaged pen needles causes significant practical difficulties and therefore often diabetics do not carry around the ideal number of replacement pen needles or, in some cases, any at all. As a result, compliance with the requirement to replace the pen needle after every use is low. Instead of removing and disposing the pen needle after every use, as advised, diabetics often simply replace the pen cap just after injection and thus store the pen with the pen needle attached. When they next require a dose of insulin, they simply remove the pen cap and reuse the same needle. The insulin pen needle creates an open connection between the insulin reservoir and its surroundings. This allows for solvent to leak when the pen is stored at elevated temperatures followed by entry of air into the insulin cartridge upon a temperature decrease. Leakage of solvent and air inside the insulin reservoir may lead to over- and under-medication, respectively.

Not replacing the pen needle after each use may be more convenient for the user, however it has been found that it is not ideal in delivering an insulin dose. Due to the fact that pen needles are extremely fine in order to allow them easily to penetrate a user's skin, they are only really intended for single use. When a pen needle is reused multiple times, the needle itself becomes deformed and even blunt. This has been found to cause increased pain, irritation and lipodystrophy to users when inserting the pen needle into their skin. Furthermore, increased lipodystrophy has been correlated with reduced insulin uptake and distribution. In spite of these factors, many diabetics still proceed to reuse needles due to the hassle they associate with having to carry multiple sterile pen needles and the disposal of used needles and associated packaging. Some users have even reported only replacing their pen needle every two to three weeks. This therefore highlights the inconvenience associated with carrying around a large number of individually packaged pen needles.

It has also been found that repeatedly using non-sterile pen needles may increase the risk of infection.

The Applicant has recognised that the problem of non-compliance of replacing pen needles needs to be addressed in order to better treat those who suffer from diabetes. A device that can conveniently store and dispense a plurality of objects such as pen needles may also find wider use.

The present invention aims to address the problems discussed above and when viewed from a first aspect there is provided storage device for storing a plurality of objects, comprising: a carousel defining a plurality of cavities; a sealing strip extending around the carousel so as to seal the plurality of cavities; a concentric guide structure extending at least partially around the carousel; and a driving member attached to the sealing strip and arranged, when moved relative to the guide structure, to pull on the sealing strip and thereby release a portion of the sealing strip and subsequently to wrap said portion around the guide structure.

It has been appreciated by the Applicant that a storage device in accordance with the first aspect of the invention would be capable of housing a plurality of pen needles suitable for attachment to an insulin pen. The plurality of objects may therefore be a plurality of pen needles stored in the cavities, in use. Storing the pen needles in such a device would mean that a user may only have to carry an insulin pen and the storage device in accordance with the invention in order to administer a large number of doses. It has been appreciated that storing a plurality of objects e.g. pen needles in a single device removes the need for a user to carry around a large number of individually packaged objects e.g. needles. By providing a device which can conveniently store a number of pen needles, the Applicant has recognised that a user may be more inclined to replace the pen needle after every use and therefore comply with recommended use of pen needles.

It has also been appreciated by the Applicant that storage devices in accordance the present invention only require a single sealing strip to seal all of their cavities. The fact that the sealing strip is wrapped around the guide structure as it is released from the carousel, so as to unseal the cavities, means that no loose waste material is produced as the device is used. A user will no longer have to worry about disposing of any waste generated from the packaging as it is used and therefore may be more likely to regularly use the device to replace the pen needle on their insulin pen.

Additionally, once a pen needle has been used, a user may easily replace it back into a cavity so that the pen needle can be safely stored within the storage device itself. The plurality of objects may therefore comprise a mixture of used and unused pen needles stored in the cavities, in use. Providing means to safely store any used pen needles may further promote the replacement of pen needles, as it was known that a lack of means to dispose of used needles is something that has previously prevented users from regularly replacing pen needles. This may also help to reduce the number of pen needles which are disposed of in general waste bins and thus potentially reduce the contamination risk for third parties.

The arrangement of the guide structure, which is positioned concentrically with the carousel, means that the guide structure may be relatively close to the carousel and thus the entire device may not need to be much larger than the size of the carousel. It has been appreciated by the Applicant that this arrangement may be particularly advantageous as keeping the storage device compact may be important in providing a device that a user can easily keep on their person, for example in a pocket on their clothing or in a handbag. Making the device easy for a user to keep on their person may increase the likelihood of them using it and thus replacing the pen needle after every time they administer a dose.

The arrangement of the guide structure, which is positioned concentrically with the carousel, may also mean that the space within the storage device is used more efficiently. This may reduce the total amount of material required to manufacture the storage device. Advantageously, storage devices in accordance with the invention may be at least partially disposable and therefore a small reduction in the amount of material required to make a single storage device may provide a significant reduction in the total amount of material required to produce a large number of them.

It has also been recognised by the Applicant that the storage devices in accordance with the first aspect of the present invention may provide a relatively simple solution which is capable of peeling away the sealing strip from the carousel and storing it in a convenient manner with a relatively small number of components and moving parts. This may make the storage device easier to operate by a user and thus further improve compliance by the user to regularly replace pen needles. Furthermore, the size and shape of the device may make changing needles easier for persons with reduced fine motor skills and also help to reduce needle stick injuries by allowing a user's fingers to be placed further away from the needle receptacle e.g. one of the cavities used to store a used needle.

It will be appreciated by those skilled in the art that as the driving member pulls on the sealing strip, it pulls away from the carousel a section of the sealing strip which was previously sealed to the carousel. It is this portion which is released from the carousel which forms the released portion. This released portion is then pulled by the driving member and wrapped around the guide structure. As will be described in more detail below, the released portion is preferably stored around the guide structure.

The driving member may gradually release the sealing strip from the carousel such that the sealing strip is pulled away in a continuous manner. Alternatively, the sealing strip may be sealed to the carousel in such a way that the driving member pulls on the sealing strip and releases portions with a predefined length, for example a portion having the length of a cavity.

The Applicant has appreciated that it may not be necessary for a driving member to pull the sealing strip around a guide structure, and thus according to a second aspect of the present invention there is provided a storage device for storing a plurality of objects, comprising: a carousel defining a plurality of cavities; a concentric guide structure extending around the carousel; wherein the guide structure comprises an opening to allow access to at least one of the plurality of cavities; and a sealing strip formed as a loop, wherein a first portion of the loop extends around the carousel to seal the plurality of cavities and a second portion of the loop extends, via the opening, around the guide structure.

A device in accordance with the second aspect of the invention may peel a sealing strip away from the carousel without the need for a driving member to pull the sealing strip. By driving relative movement between the guide structure and the carousel, e.g. by rotating the carousel, the sealing strip may be pulled away from the carousel so as to reveal cavities. It will be understood that due to the sealing strip being formed as a loop, as a first portion of sealing strip is released from the carousel and pulled around the guide structure, a second portion of the sealing strip comes around and passes back on to the carousel. This process may occur up to the point at which sealing strip which was sealed to the carousel has been peeled away so as to remove the initial seal.

The portion of the loop which comes back around on to the carousel may not fully seal back to the carousel to the extent that the sealing strip was previously sealed, however the tension in the system may mean that it is held sufficiently tightly against the carousel such that it effectively reseals the carousel. As mentioned above, the plurality of objects may be a plurality of pen needles, optionally comprising a mixture of used and unused pen needles, stored in the cavities in use. The sealing strip being formed as a loop provides a convenient way of resealing cavities that may contain used needles. Thus it is preferable that the second portion of the loop wraps around the guide structure and subsequently extends back around the carousel to reseal one or more of the plurality of cavities.

The invention according to the second aspect of the invention provides many of the same advantages related to improved compliance as those described above in relation to the first aspect of the invention.

In embodiments according to either aspect of the invention, the storage device may store a plurality of pen needles and all of the pen needles in their respective cavities using a single sealing strip. The pen needles may be sealed in their respective cavities using a single sealing strip. Due to the fact that the cavities are sealed by the sealing strip it may not be necessary for the pen needle to have any other protective packaging other than the cavity and the sealing strip. By storing the pen needles in this manner, the total amount of packaging required to store the needles may be reduced. Furthermore it may be possible to attach the pen needle to the pen whilst the pen needle is housed within the cavity in the carousel. Attachment in this way may mean that the pen needle does not require the separate cap which covers the needle itself as the risk of a user pricking themselves is reduced. This may therefore further reduce the amount of protective packaging required.

In one set of embodiments, in accordance with the second aspect of the present invention, the storage device further comprises a driving member arranged to cause relative movement between the carousel and the guide structure. Whilst it may be possible to cause movement between the carousel and guide structure without a driving member, the Applicant has appreciated that a drive member may make causing relative movement easier and thus make the device easier to use. It will be appreciated that the driving member in this set of embodiments does not act to pull directly on the sealing strip, as with the driving member in accordance with the first aspect of the present invention, rather that it is used to drive relative movement between the carousel and guide structure so as to cause the sealing strip to be released from the carousel.

In a set of embodiments in accordance with either aspect of the invention, the driving member comprises a planar member that extends in a plane substantially parallel to the plane of the carousel. The planar member may, for example, comprise a continuous surface. Alternatively, the planar member may have cut-out portions. The cut-out portions may effectively define spokes which may extend from the centre of the planar member towards an outer edge of the planar member.

Alternatively the driving member could simply comprise a rotating arm extending from a centre of the device.

In another set of embodiments the driving member is not connected to a centre of the device to permit rotation. It could, for example, be guided for rotation by a slot in the housing through which a portion of the driving member extends.

In a further set of such embodiments in accordance with the first aspect of the invention, the driving member comprises an extending portion which extends in an axial direction with respect to the carousel. The extending portion may be arranged to engage the sealing strip. By having a portion which extends axially, it may be easier for the driving member to pull and direct the sealing strip around the guide structure.

Depending on the configuration of the planar member and the guide structure, the extending portion may extend from any appropriate section of the planar member in embodiments wherein the driving member comprises a planar member. However, in one set of embodiments, the extending portion extends from an edge of the planar member. By having a portion extending from the edge of the planar member, it may be easier for a user to attach the sealing strip to the extending portion when assembling the storage device, and it may be easier for a user to position the planar member, when assembling the storage device, such that the extending portion passes around the guide structure. In such embodiments the driving member may comprise a driving cover that is a planar member extending in a plane substantially parallel to the plane of the carousel, and an extending portion which extends in an axial direction from an edge of the driving cover.

In one set of embodiments the guide structure follows a circular arc with a first radius. A device which has a circular configuration may be particularly advantageous as the device may remain relatively simple and also it may assist in ensuring consistently smooth motion throughout the entire range of motion of a/the driving member. In such a set of embodiments, preferably the extending portion follows a circular arc with a second radius, wherein the second radius is larger than the first radius. This may mean that the extending portion can conform to the shape of the guide structure and this may therefore assist in keeping the device relatively compact. Whilst it may be advantageous in certain instances for the device generally to have a circular shape, the Applicant has recognised that this may not always be the case and has appreciated that the various structures of the storage device may have any appropriate shape that permits relative movement between them.

A compact device, e.g. having a circular configuration, may be desirable in embodiments according to either aspect of the invention. In embodiments according to the first aspect of the invention, the portion of the sealing strip that wraps around the guide structure may be stored around the guide structure. The guide structure may therefore act as a take-up or storage means e.g. storage reel for the sealing strip. Preferably the device does not include a separate take-up or storage reel. In a preferred set of embodiments, the released sealing strip is stored concentrically around the carousel. In embodiments according to the second aspect of the invention, the second portion of the loop extends around the guide structure to wrap around the guide structure. The second portion of the loop is preferably wrapped around the guide structure until it meets the first portion of the loop extending around the carousel. In a preferred set of embodiments, the loop extends concentrically around the carousel. Preferably the loop does not extend around a separate take-up or storage reel. All of these embodiments provide the benefit of a compact take-up configuration for the sealing strip after it has been released from sealing one or more of the cavities of the carousel. The overall footprint of the device may not be much larger than that of the carousel. In other words, the size of the device is not dictated by the mechanism that moves the sealing strip but by the size of the carousel and its cavities. This can also reduce the material cost of the device.

In embodiments according to either aspect of the invention comprising a driving member, the driving member may comprise an external surface which may be engaged by a user. This facilitates the user during relative movement between the carousel and the guide structure. In a further set of embodiments the external surface comprises a plurality gripping features. These gripping features may be engaged by a user when operating the device. The gripping features may be any features which provide improved grip of the external surface. For example, a non-exhaustive list of gripping features may include: protrusions, roughened sections, additional materials attached to the external surface e.g. rubber, scalloped sections.

The driving member and the carousel and/or guide structure may all be considered to move relative to one another in a rotary fashion. There are various means through which this rotary motion may be achieved, however according to a set of embodiments the driving member comprises a cylindrical structure extending from its centre to form part of a bearing to facilitate motion around the carousel and/or the guide structure. Such a cylindrical structure may not only facilitate motion but may also make assembly of the device easier. The cylindrical structure extending from the guide structure may engage with a second part which may be operatively connected to the carousel and/or guide structure.

In embodiments according to either aspect of the invention comprising a driving member, the driving member is preferably arranged to move concentrically around the carousel. The driving member may be arranged to rotate around an axis passing through the centre of the carousel. For example the driving member may comprise a cylindrical structure, as mentioned above, that is mounted to rotate around an axis passing through the centre of the carousel. The driving member may be rotated up to 360° around the carousel, for example causing the sealing strip to be fully wrapped around the carousel and concentric guide structure.

In addition, or alternatively, the carousel, guide structure and driving member may all be arranged to move relative to one another in a rotary fashion around a common axis. Preferably the common axis is a central axis of the carousel. The carousel, guide structure and driving member may all have the same circular configuration. The diameter of the guide structure may be slightly larger than the diameter of the carousel, and the diameter of the driving member may be slightly larger than the diameter of the guide structure. In one or more of these embodiments, the portion of the sealing strip that wraps or extends around the guide structure may be stored along a circumference outside the guide structure. The driving member may engage the inside of the sealing strip, i.e. between the sealing strip and the guide structure, or the driving member may engage the outside of the sealing strip, i.e. on the opposite side of the sealing strip to the guide structure. It will be appreciated that such embodiments provide a compact storage device having the driving member operating close to the carousel and guide structure.

In a set of embodiments the storage device comprises a ratchet arranged to restrict relative movement between the driving member and/or the carousel and/or the guide structure. This may be arranged to ensure that the carousel only moves in one direction such that only new cavities are revealed, rather than being able to access previously unsealed cavities. There may be many ways that this can be achieved, however in a set of embodiments the cylindrical structure comprises part of a ratchet. There may be individual ratchet mechanisms arranged to restrict relative movement between the various components. In one set of embodiments a ratchet mechanism operates between the guide structure and the driving member. In another set of embodiments a ratchet mechanism operates between the driving member and the carousel. In a further set of embodiments a ratchet mechanism operates between the driving structure and the carousel. A ratchet system may be particularly advantageous when the device is used to store pen needles. The ratchet system may prevent reverse motion of the carousel and therefore only permit the user to access sterile pen needles contained in unopened cavities.

The Applicant has recognised that due to the fact that many diabetics have to administer insulin doses so regularly throughout the day, many users always carry a pen needle on the end of their insulin pen in order to ensure that they will always be able to administer a dose irrespective of whether they have any fresh, sterile, pen needles available. In one set of embodiments, intended to counter this perceived need, the device is arranged such that it is possible to overcome the ratchet mechanism and allow reverse movement of the carousel so as to reveal a previously unsealed cavity which may contain a used needle. Additionally, the guide structure could be manually moved by sliding the guide structure relative to the carousel so as to present the last used needle and thereby avoids the motivation for some users to store a used needle on the pen. This may therefore allow a user still to administer an insulin dose although all pen needles within the cavity have been used. This feature may therefore provide the security of always having an accessible needle for a user and there may encourage them to use such a device and be more compliant with the recommendation to replace their pen needle regularly.

In a set of embodiments in accordance with either aspect of the invention, the driving member forms part of a cover for the storage device. It may therefore act to cover the carousel and other components, i.e. a driving cover. In another set of embodiments, the guide structure is provided on a base part of the storage device which acts to house other parts of the storage device, for example the carousel. The base part may therefore be considered to be a base cover for the device and therefore in a set of embodiments the guide structure is formed as an integral part of the base cover. In a further set of embodiments the base part comprises a closed base from which the guide structure extends. In another set of embodiments, the driving member which forms part of a cover interacts with the base part, which comprises the guide structure, so as to form an enclosed storage device.

In a set of embodiments in accordance with the second aspect of the invention, wherein the device comprises a driving member, the guide structure may be provided on the driving member. For example, the guide structure may be a guide wall extending from a driving cover.

The concentric guide structure may take any form that is suitable to guide the sealing strip. In one set of embodiments the guide structure is formed of at least one guide wall which extends at least partially around the carousel. As the sealing strip may be in the form of an elongate strip or loop, a guide wall may be particularly suitable for guiding the sealing strip around the carousel.

The guide wall may comprise one or more features arranged to ensure a smooth passage for the sealing strip, especially for a portion of the sealing strip or loop as it first comes into contact with the guide wall. In one set of embodiments, a rounded end is formed on one edge of the guide wall. The rounded end may comprise a roller, for example a cylindrical roller, to reduce friction between the sealing strip and the edge of the guide wall. This may be particularly important in embodiments of the second aspect of the invention wherein the loop makes a turn of up to 180° around the end of the guide wall. In addition, or alternatively, in one set of embodiments an edge or end of the guide wall is provided with a low friction surface.

It may be important for the storage device to operate in a smooth manner and therefore the guide structure may be provided with a low friction surface which allows the sealing strip to pass along the guide structure easily. A low friction surface, i.e. one reducing friction between the guide structure and the sealing strip, may be achieved in various ways. Such a low friction surface may be achieved by an appropriate surface finish (e.g. a dimpled surface like a golf ball) and/or surface coating (e.g. PTFE or other lubricant material) and/or reduced surface area in contact with the sealing strip (e.g. a stepped or ridged surface). In addition, or alternatively, the guide structure may comprise one or more rollers or bearings arranged along the guide structure to ease the passage of the sealing strip around the guide structure. The Applicant has found that considerable friction can build up between the sealing strip and the guide structure as an increasing portion of the released sealing strip wraps around the guide structure, for example when unsealing the second half of cavities in the carousel.

In addition, or alternatively, the sealing strip may be provided with a low friction surface which allows the sealing strip to pass along the guide structure easily. The material of the sealing strip and of the guide structure may be chosen to ensure a low coefficient of friction between the material pair.

In one set of embodiments the guide structure is formed from a single, continuous, guide wall. This may help ensure that the released sealing strip is isolated from the carousel and assist in preventing any cross contamination. However, the guide structure may not necessarily be in the form of a continuous structure and therefore in an alternative set of embodiments the guide structure is formed of a plurality of discrete wall portions. If the discrete wall portions are positioned appropriately it may still be possible to properly guide the sealing strip around the wall portions. Such a set of embodiments may reduce the total amount of material required to manufacture the device and thus may reduce the cost. In either of the above sets of embodiments the wall/wall portions may have cut-outs or apertures.

In embodiments wherein the guide structure is formed of a plurality of discrete wall portions, the Applicant has recognised that friction between the guide structure and the sealing strip can be reduced. A low friction guiding process may be further enhanced by providing one or more of the discrete wall portions with a low friction surface and/or one or more rollers or bearings, as mentioned above. In one set of embodiments, the discrete wall portions may each comprise a rounded end formed on one or both edges. The rounded end may comprise a roller, for example a cylindrical roller, to reduce friction between the sealing strip and the edge of the guide wall portion.

In a set of embodiments the guide structure extends at least 180° around the carousel, preferably at least 270° around the carousel. Having a guide structure which extends substantially around the carousel may ensure that any released sealing strip is held away from the carousel, ensuring that the device is easy to operate. It will be appreciated that the larger the angular extent of the guide structure, the more important it may become to avoid a build-up of friction as the sealing strip wraps around the guide structure.

It will be appreciated by those skilled in the art that in accordance with either aspect of the invention, the carousel may effectively be surrounded by the guide structure. Therefore, in one set of embodiments, in order to gain access to the plurality of cavities defined by the carousel, the guide structure comprises at least one opening. Depending on the particular application of the device there may be a plurality of openings, for example if it is necessary to extract an object from two different cavities simultaneously. This may be relevant, for example, when a device is used to store and dispense drugs.

In a further set of embodiments the opening has a suitable dimension to fully expose no more than one of the plurality of cavities at any one time. Sizing the opening in this manner ensures that an object which fills the cavity can easily be removed. Additionally, it means that it is not possible to unseal/access two cavities in the same opening at the same time. This may mean that only one cavity is unsealed at any one time, which is particularly relevant when the device is used to store and dispense pen needles as it will be appreciated that it would be undesirable to unseal sterile pen needles prematurely as they could then become contaminated prior to use.

It will be appreciated by those skilled in the art that a released/loose portion of the sealing strip may effectively pass from the carousel around an edge of the guide structure, possibly at its opening, and then around the guide structure. In one set of embodiments an end of the guide structure which defines the opening has an at least partially rounded edge. Passing the sealing strip around a rounded edge may mean that the sealing strip passes more easily around the corner defined by the edge of the guide structure and this may result in the device operating in a smooth manner. A rounded edge may also prevent inadvertent damage occurring to the sealing strip which may otherwise be caused by a sharp edge. In a further set of embodiments the at least partially rounded edge comprises a roller which is free to rotate about an axis. The Applicant has recognised that by providing a rounded edge which is provided by a roller the sealing strip may pass even easier past the corner, potentially making the device even easier to use. As a certain minimum force will be required to peel the sealing strip away from the carousel, the smoother the sealing strip passes along other components, the easier the device may be to use and thus the smaller the force required by a user to operate by the device. Again, making the device easier to use may help to improve user compliance in cases where it is used to store pen needles for diabetics.

The carousel may take any suitable form that allows the sealing strip to be peeled away from the carousel and pass around the guide structure. However, in a set of embodiments the carousel has a circular structure. In a set of such embodiments the carousel comprises an aperture positioned at its centre. In a further set of embodiments each of the plurality of cavities extends radially away from a centre of the carousel. This particular arrangement may provide for a relatively simple storage device which is easy for a user to operate.

As discussed above, the device may be used to store and dispense a wide variety of different objects. The particular use of the storage device may dictate the number of cavities which are required in the carousel. However, in a set of embodiments the carousel comprises at least 7 cavities, preferably at least 10 cavities, more preferably at least 12 cavities. It has been appreciated by the Applicant that for some applications, if the number of cavities is too low then the advantage of using the storage device may be lost.

In accordance with embodiments of the first aspect of the invention, the device may be particularly well suited to unsealing the cavities and storing the sealing strip, however, it may not necessarily have any means of effectively resealing the unsealed cavities. In certain applications the Applicant has appreciated that it may be desirable to closely re-cover the cavities, for example, when the device is used to store and dispense insulin pen needles. It is perceived that pen needles have an undesirable odour once they have been used, due to residual insulin which remains on the pen needle. If the used pen needles are stored in the storage device, it would be desirable to limit the ability of smell to emanate from the cavities which contain used pen needles. Addressing this issue may mean that a user is more likely to remove and store used pen needles in the device thus potentially improving their compliance with the recommendation of regularly replacing their pen needle.

Depending on the particular arrangement of the components of the storage device, the unsealed cavities may effectively be resealed as a result of the various components being a tight fit. However in a set of embodiments the device further comprises a band that at least partially surrounds the carousel. Such a band may form a close fit with the carousel such that it acts to seal any cavities from which the sealing strip has been removed. The band may not seal the cavities to the extent that the sealing strip seals the cavities however it may be sufficient, for example, to prevent odours from escaping the cavities. The band preferably extends around the carousel, between the carousel and the concentric guide structure.

To achieve a tight fit with the carousel, in a set of embodiments the band has a natural radius which is smaller than the radius of the carousel and is at least partially resilient so as to grip onto the carousel when placed around the carousel.

In a further set of embodiments the band may comprise at least one rim and the carousel may comprise a complementary groove. These complementary features may assist in locating the band and guiding its motion as well as further improving the seal between the band and the carousel.

The tight fit between the band and the carousel may have a secondary advantage. When the device is operated, and the sealing strip is peeled away from the carousel, this may cause the carousel to rotate relative to guide structure. In embodiments where there is a tight fit between the band and the carousel, as the carousel rotates this may cause the band to move with the carousel and therefore also to rotate. The advantage of this associated movement will become apparent when discussed in the context of a further set of embodiments discussed below.

In a set of embodiments the band comprises a protrusion extending into a/the opening. Such a protrusion may make it possible for a user to easily slide the band relative to the carousel in order to close the opening. In other words it allows the band to act as a shutter. This may be advantageous in reducing any contamination of the device. Additionally, it may be the case that at least one cavity is always unsealed and present at the opening, therefore by providing a shutter it may be possible to close off the opening and prevent access. This may be useful when the device is used to store pen needles, as it may avoid having a used pen needle being exposed.

In a further set of embodiments the moveable shutter is provided by a/the band and preferably a/the protrusion may be moved within the opening so as to achieve the closing of the opening. The Applicant has recognised that the band can act to both seal the cavities and close the opening. As mentioned above, it is when the band is used to close the opening that the tight fit between the band and the carousel may become apparent. For example, the device may be in a configuration in which the band is in a position which closes the opening. A user may then want to proceed to extract an object from one of the sealed cavities. A user may move the band, optionally by applying a force to the protrusion discussed above, such that the opening is revealed and then drive the device such that the sealing strip is removed and an unsealed cavity is presented at the opening. Alternatively, instead of first moving the band, a user may proceed directly to driving the device. Due to the tight fit between the band and the carousel, as the sealing strip is peeled away and the carousel is caused to rotate the band may rotate in accordance with the carousel and move to reveal the opening. The band may move and an unsealed cavity may be presented simultaneously. The Applicant has recognised that this may be a simple means of accessing a new pen needle. Once again making the device as simple as possible to use may further improve the chance of a user actually regularly using the device.

Other forms of shutter can also be envisaged, not necessarily provided by a band. More generally therefore in embodiments where the guide structure comprises an opening, the device may comprise a moveable shutter arranged to close the opening.

The moveable shutter may be provided by any appropriate means arranged to close the opening. For example, the shutter may comprise: a hinged lid, a sliding door, sealing tape, a silicon seal that may be moved or a seal that can be squeezed through.

In a set of embodiments the storage device is arranged to present information to the user regarding the status of the storage device. For example by appropriately arranging information along the length of the sealing strip it may be used to provide relevant information, for example relating to the number of cavities which have been unsealed, or the number of cavities which remain sealed. Therefore, in a set of embodiments the sealing strip bears an indication of the number of the plurality of cavities which remain sealed or unsealed. The sealing strip may provide other indications for example by having a variation in colour along its length to provide a further visual indication to the user of the status of the device. Of course there is a wide variety of information which may be provided on the sealing strip. Depending on the particular configuration of the storage device, the information may be printed on the inside of the sealing strip, i.e. the surface which is sealed against the carousel, such that as the sealing strip is peeled away it reveals the information. Alternatively, or in addition, information may be printed on the outside of the sealing strip. Of course there may be other means for displaying information to a user for example a counter which increases incrementally as the storage device is operated.

In simple form a storage device in accordance with the invention may have only a guide structure which partially surrounds the carousel and no further outer structure. When the sealing strip passes around the guide structure it may therefore be directly visible. However, it is envisaged that in other embodiments additional outer structures are provided. In a set of embodiments, an outer part of the device is formed from an at least partially transparent material. The material may for example be transparent or translucent. It will be appreciated that a user may be able to see any information on the sealing strip through this part. It may not be necessary for all of an outer part to be formed of an at least partially transparent material; the at least partially transparent part may be in the form of a window.

In a set of embodiments the storage device further comprises an outer perimeter wall that extends at least partially around the guide structure.

In order to gain access to the cavities within the carousel, as discussed above, there may be an opening in the guide structure. In embodiments comprising an outer perimeter wall, there may also be a need to access the carousel through the outer wall. Therefore in a further set of embodiments, the outer perimeter wall defines an opening with a suitable dimension to fully expose no more than one of the plurality of cavities at any one time. The opening on the outer perimeter wall and the opening on the guide structure may be permanently aligned such that a user may access a cavity which is aligned with the openings. However, in an alternative set of embodiments, the opening on the outer perimeter wall may be moveable relative to the opening on the guide structure. By moving the openings relative to one another it may be possible to close the device and prevent access. This may also act to seal the device once a used needle had been placed into it for example with many of the advantages as discussed above in relation to re-sealing.

In embodiments which comprise an outer perimeter wall, the outer perimeter wall and the guide structure may form an annular slot. In a set of such embodiments in accordance with the first aspect of the invention the extending portion of the driving member may be located in an annular slot between the outer circumferential wall and the guide structure. This outer wall may therefore act to guide the driving member and it may also act to protect it.

Depending on the arrangement of the guide structure and also depending on how the sealing strip is sealed to the carousel, it may not be possible to seal all of the cavities. This may be the case with the second aspect of the invention. Therefore in a set of embodiments, at least one of the plurality of cavities is sealed with a separate, independent seal. It may be possible to arrange the carousel such that this individually sealed cavity is the first cavity which is revealed when the device is first used. A user may then simply remove this first seal and the device may then continue to be used with the automatic removal and storage technique as described above.

The sealing strip may be attached to the carousel by any suitable means. For example, the sealing strip may be welded to the carousel or attached using an adhesive.

The Applicant has recognised that certain components of the storage device may be used by the user more than once. The Applicant has realised that when used to store and dispense pen needles, it may be advantageous if the user can easily remove and replace the carousel in which the cavities have been unsealed with a new, sealed, carousel comprising a new set of sterile needles. In this case, a user may therefore be provided with a plurality of sealed carousels. Such a carousel is novel and inventive it its own right and thus when viewed from a third aspect, the present invention provides an insert for a storage device, comprising: a carousel defining a plurality of cavities; and a band which extends substantially around the carousel.

The insert may therefore provide a refill for a storage device. By reusing certain components of the storage device it may be possible to reduce the total cost of storing and dispensing a number of objects. Advantageously the cavities may each contain a needle. A user may simply position the insert into a housing which forms or includes a driving member as hereinbefore described and attach the end of the sealing strip to it.

In a set of embodiments the insert comprises a sealing strip which extends around the carousel so as to seal the plurality of cavities.

In a set of embodiments the band has a natural radius which is smaller than the radius of the carousel and is at least partially resilient so as to grip onto the carousel when placed around the carousel. A band according to these embodiments may have similar advantages to those previously discussed in relation to bands around the carousel (e.g. effectively re-sealing opened cavities).

In a set of embodiments the band comprises a protrusion.

The insert may be made of relatively inexpensive materials whereas the housing may be made from a more durable material as it is intended to be re-used.

In accordance with the foregoing aspects of the invention, whilst the sealing strip may be wrapped around a concentric guide structure which partially surrounds the carousel, this may not always be necessary. It may be possible to wrap the sealing strip back around the carousel itself, therefore when viewed from a further aspect the present invention provides a storage device for storing a plurality of objects, comprising: a carousel defining a plurality of cavities; a sealing strip extending around the carousel so as to seal the plurality of cavities; a guide; and a driving member attached to the sealing strip and arranged, when moved relative to the guide, to pull on the sealing strip and thereby release a portion of the sealing strip and subsequently to wrap said portion around the carousel.

According to this aspect of the invention, the released portion of the sealing strip may wrap around the carousel itself. This aspect of the invention shares many of the advantages of the earlier aspects of the invention as discussed above. The Applicant has also appreciated that many of the features discussed above in respect of the earlier aspects of the invention may also equally apply to this aspect of the invention. Thus, in embodiments according to this further aspect of the invention, one or more of the features described hereinabove may be applied alone or in combination to a device having a guide in place of a concentric guide structure.

The choice of material for the sealing strip and the particular application and size/configuration of the device may affect the ability for the storage device to wrap the sealing strip around the carousel. The Applicant has recognised that wrapping the sealing strip around the carousel itself may cause a significant frictional force between the released sealing strip being wrapped around the carousel and the carousel itself. This force may act to prevent the carousel from moving and thus prevent the unsealing of further cavities. This may be overcome by providing a sealing strip that is sufficiently smooth such that it easily passes back along the carousel and the sealing strip may only be relatively weakly sealed to the carousel to make it easier to release.

Whilst the storage device according to any aspect of the present invention may be particularly well suited to storing pen needles, the Applicant has recognised that the storage device may be suitable for storing a wide variety of different objects which require that they are stored in a sealed cavity. A non-exhaustive list of possible objects which the device may be suitable for containing includes: pharmaceutical products, food products, medical devices or small sterile parts/devices to be loaded onto a sterile field.

Some preferred embodiments of the present invention will now be described, by way of example only, and with reference to the accompanying drawings, in which:

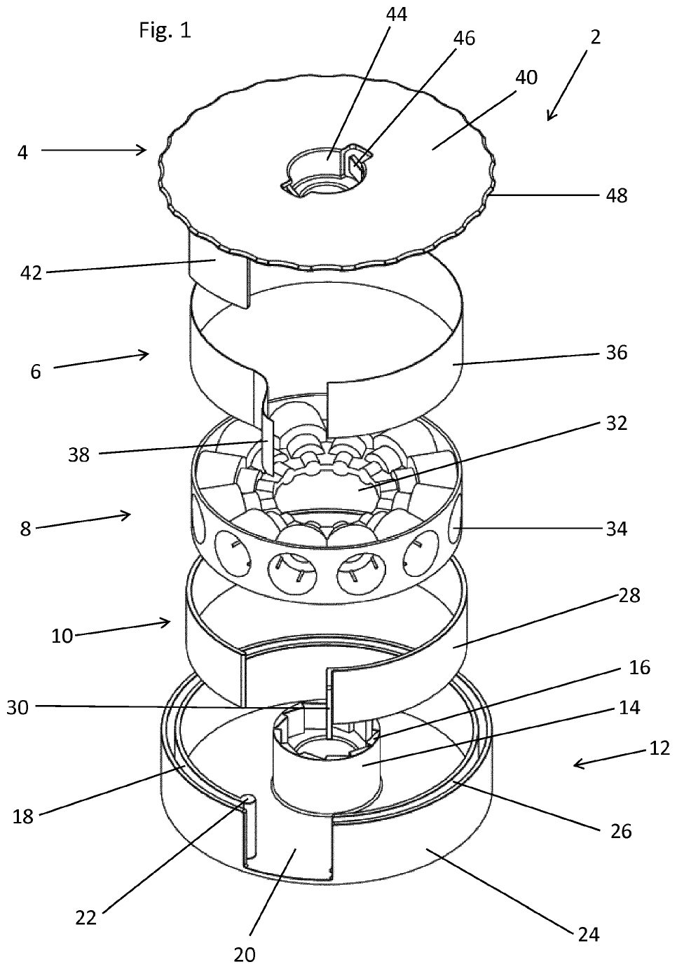

FIG. 1 illustrates a first embodiment of a storage device in accordance with the present invention;

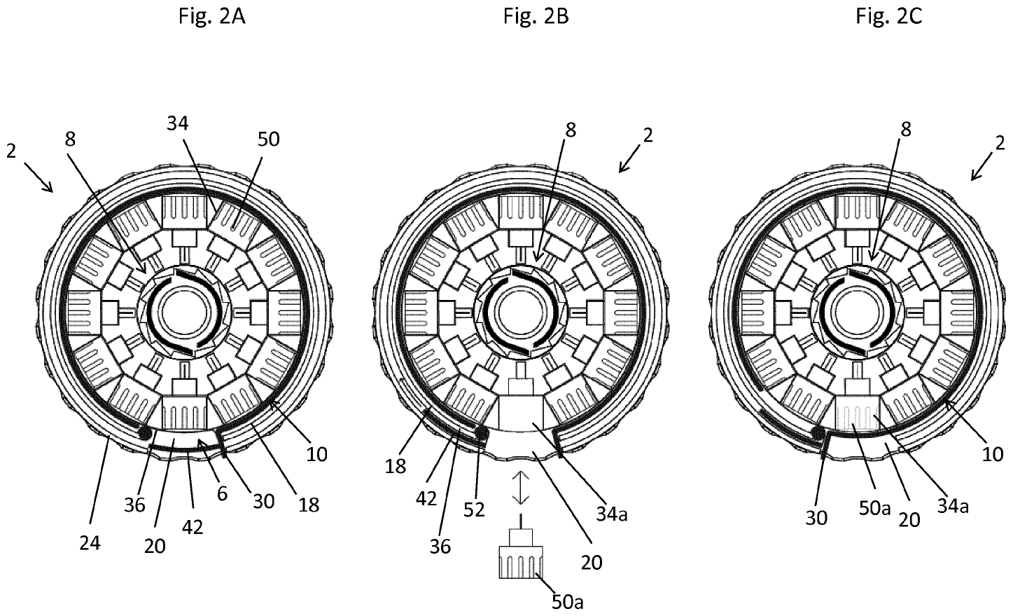

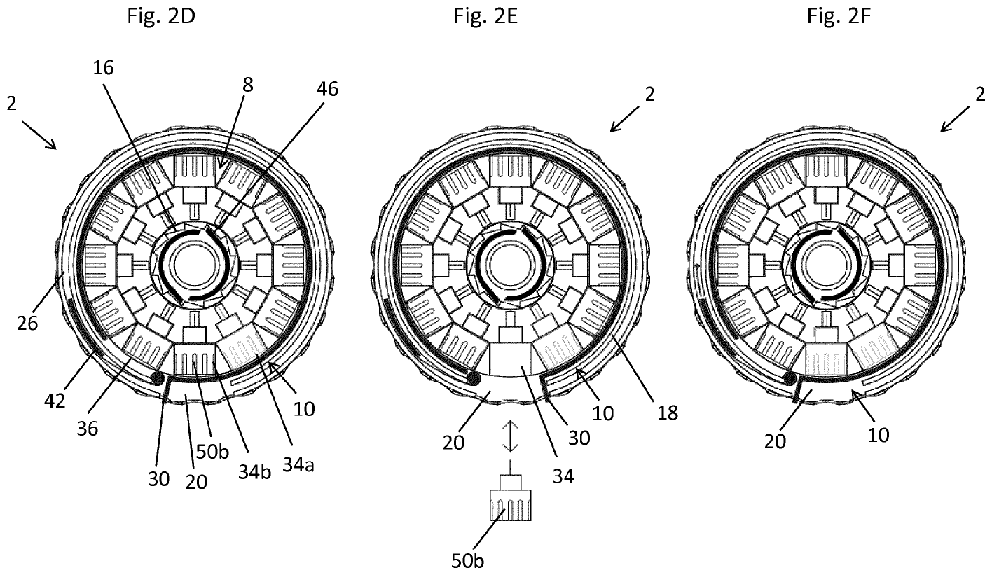

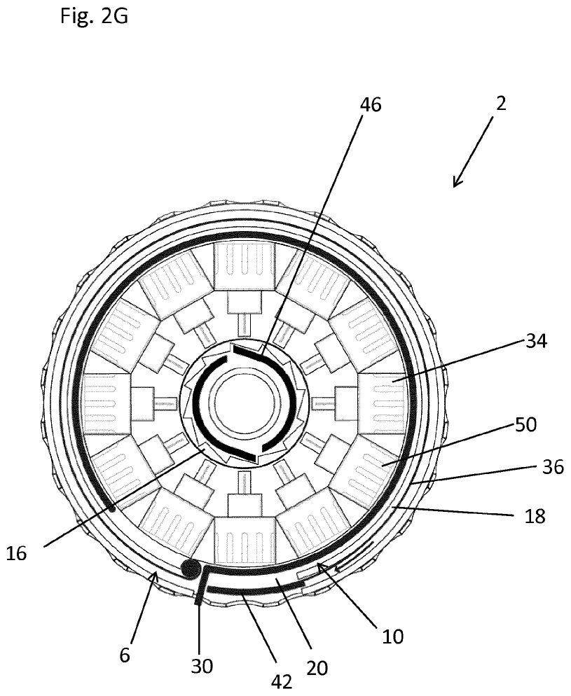

FIGS. 2A-2G are sectional views illustrating operation of the storage device seen in FIG. 1;

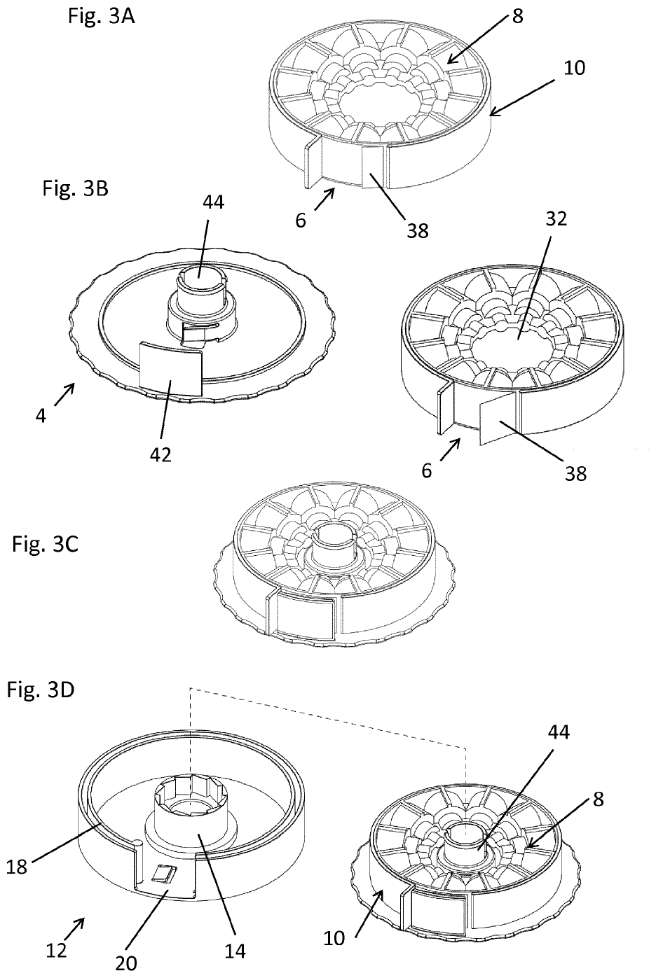

FIGS. 3A-3D illustrate the assembly of the storage device seen in FIG. 1;

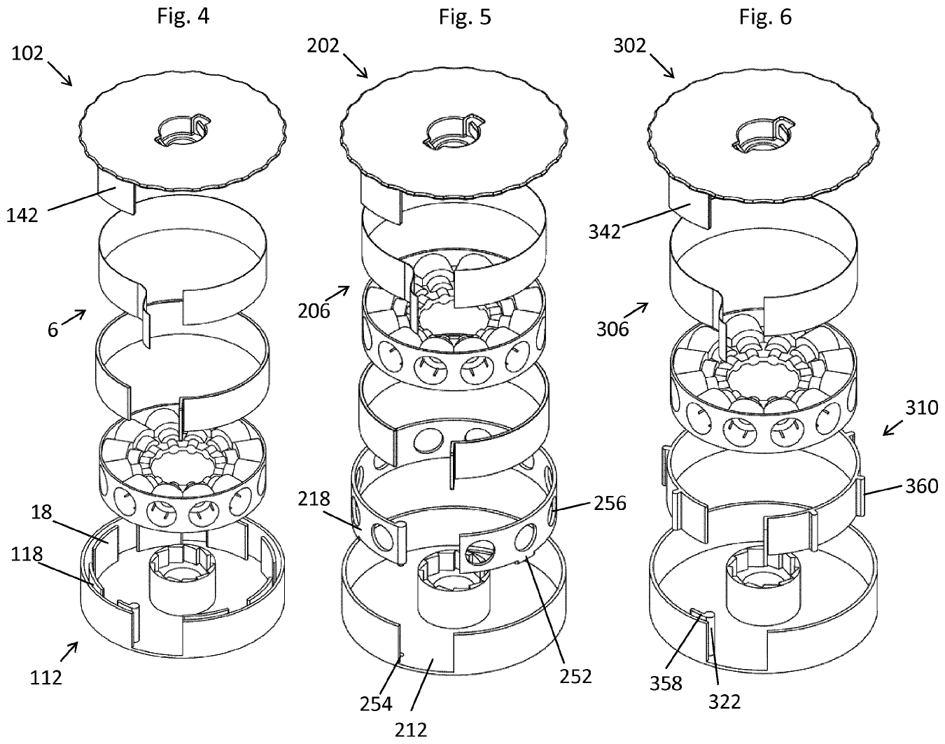

FIG. 4 illustrates a second embodiment of a storage device in accordance with the present invention;

FIG. 5 illustrates a third embodiment of a storage device in accordance with the present invention;

FIG. 6 illustrates a fourth embodiment of a storage device in accordance with the present invention;

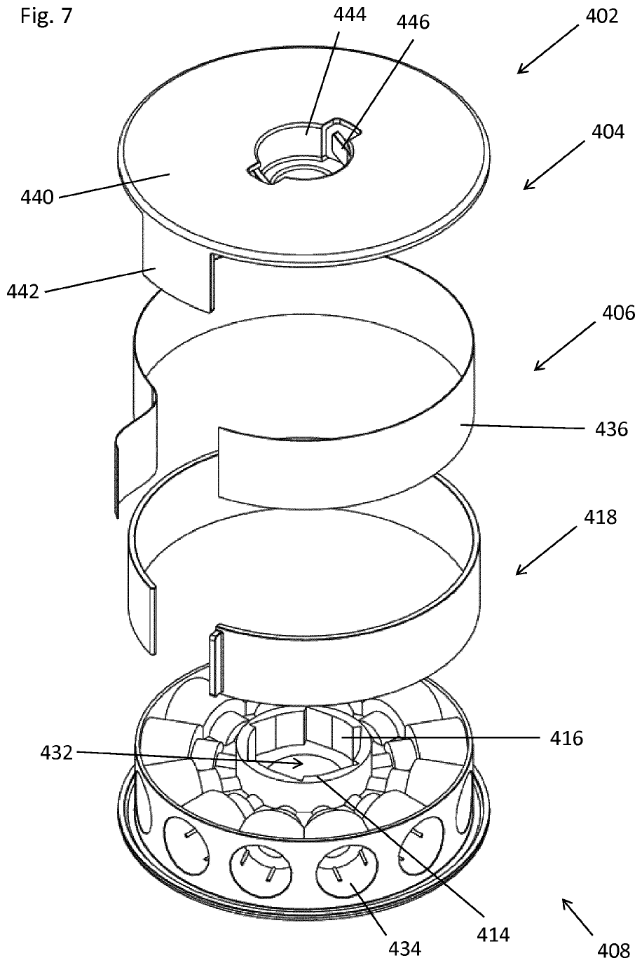

FIG. 7 illustrates a fifth embodiment of a storage device in accordance with the present invention;

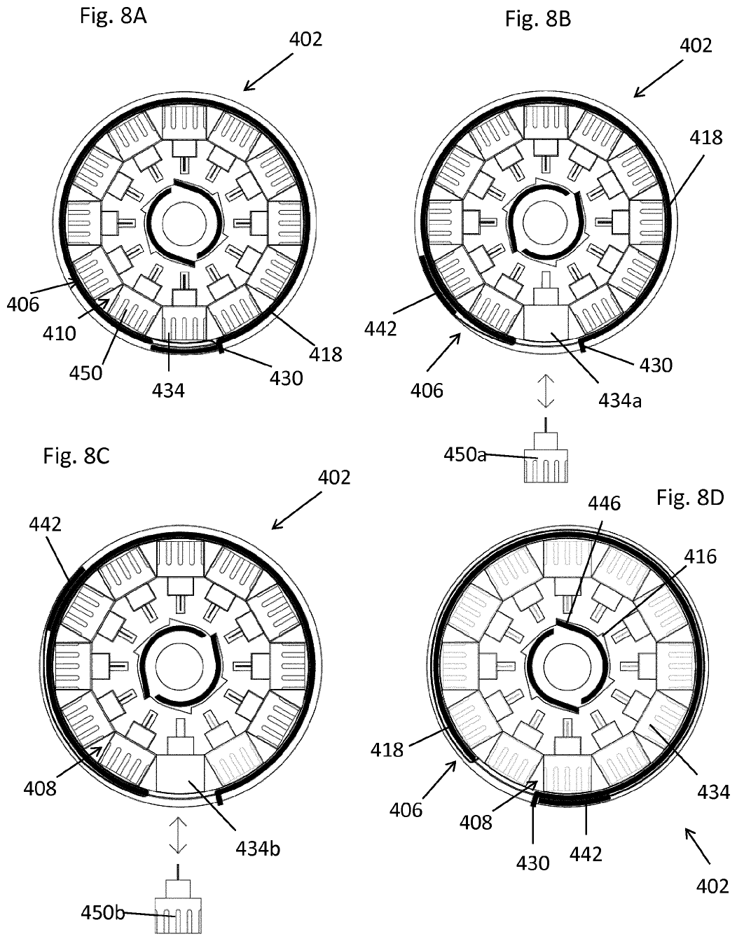

FIGS. 8A-8D illustrate the operation of the storage device seen in FIG. 7;

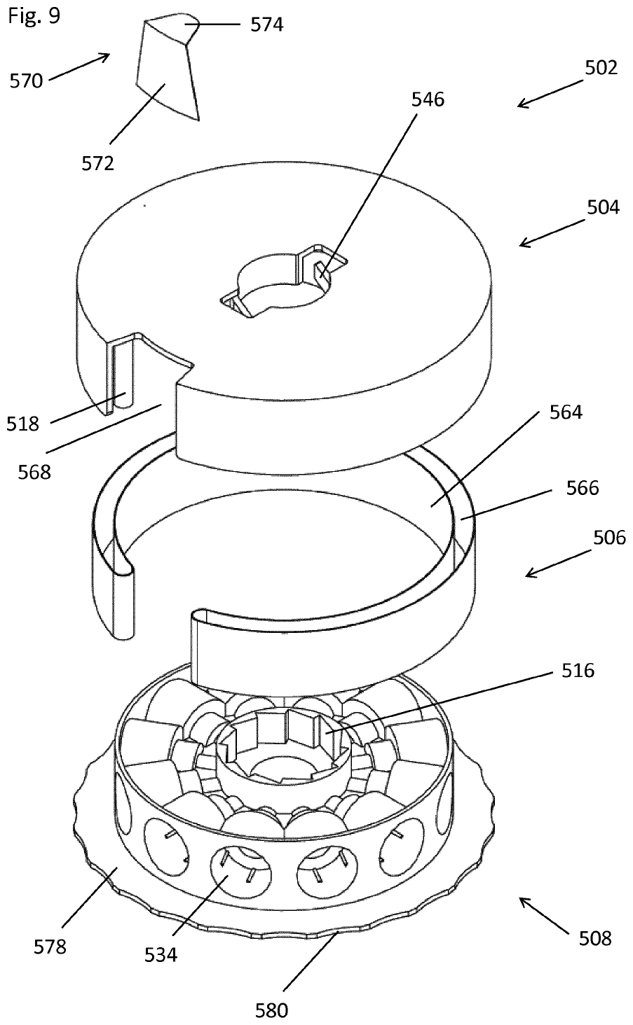

FIG. 9 illustrates a sixth embodiment of a storage device in accordance with the present invention;

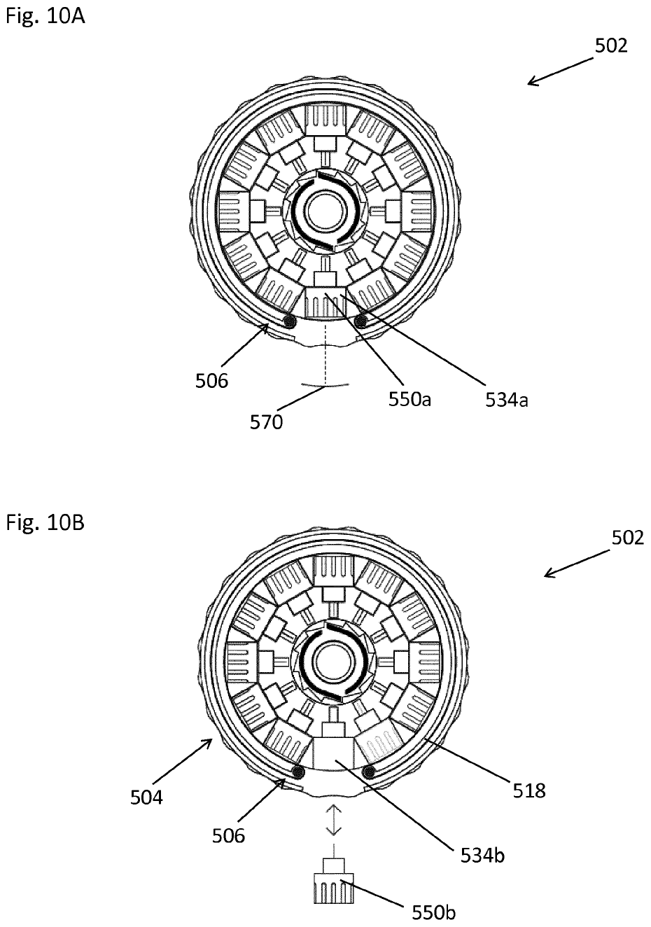

FIGS. 10A-10B illustrate the operation of the storage device seen in FIG. 9;

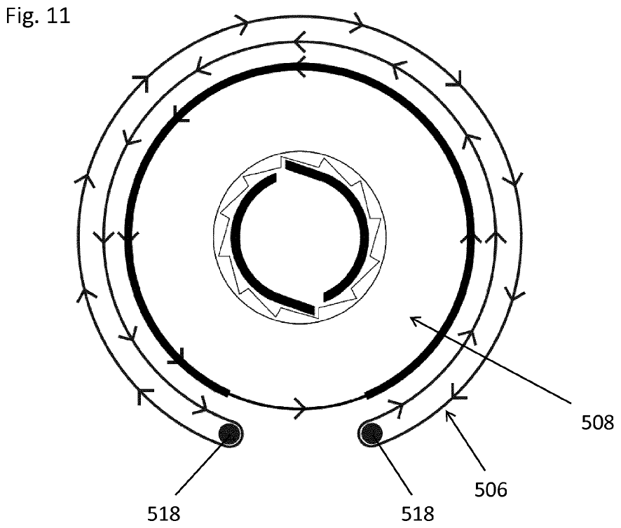

FIG. 11 illustrates the sealing strip of the storage device seen in FIG. 9;

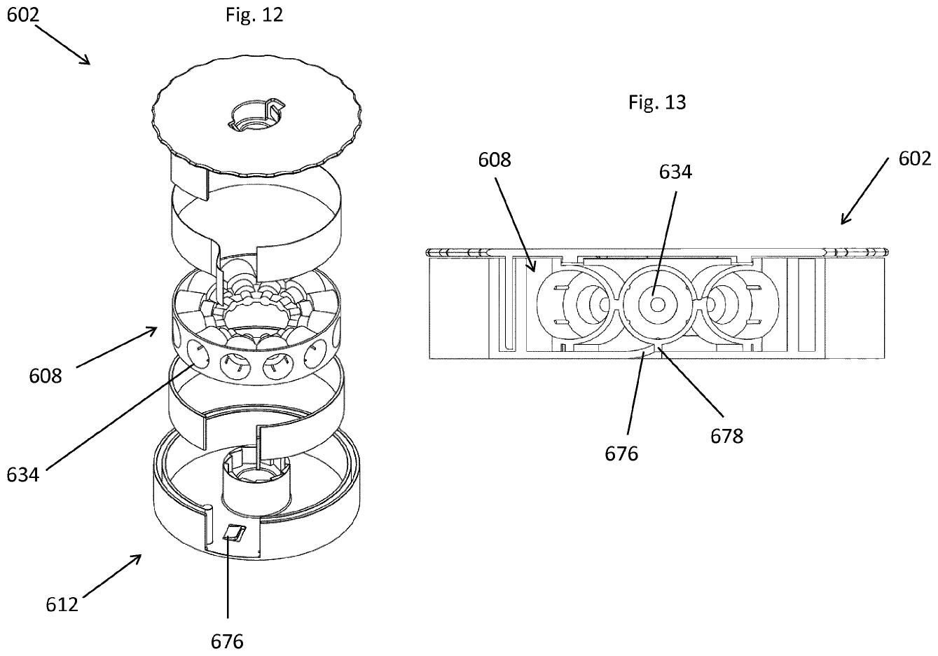

FIG. 12 illustrates a seventh embodiment of the invention comprising a carousel ratchet;

FIG. 13 is a cross-sectional view of the storage device seen in FIG. 12;

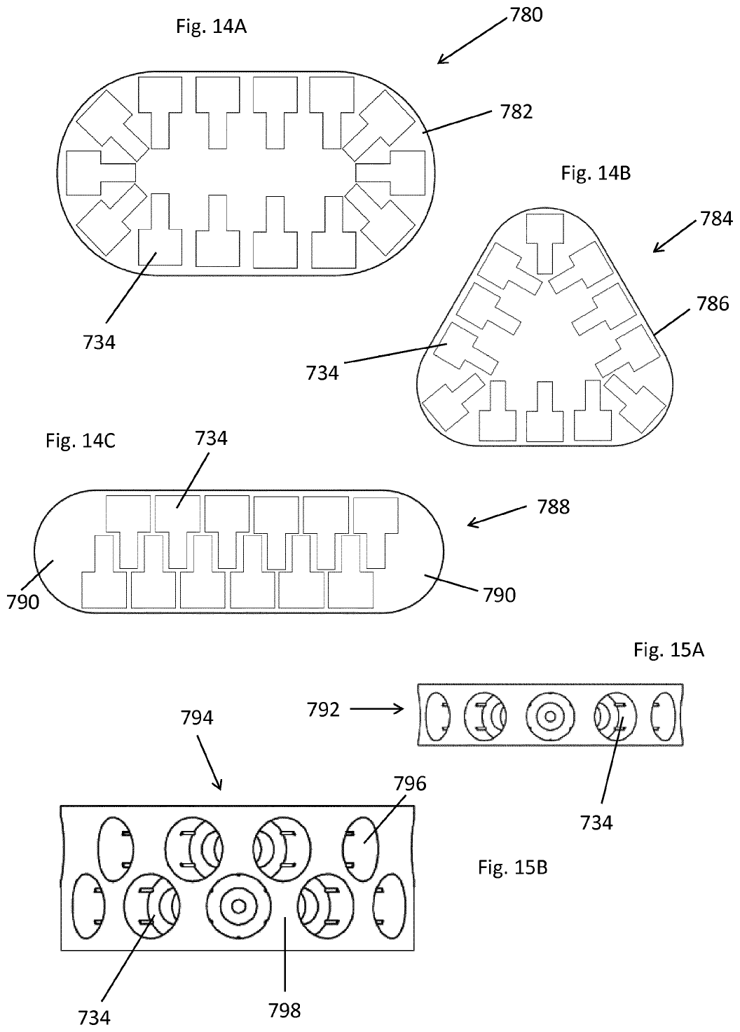

FIGS. 14A-14C illustrate further embodiments having different shapes of the device;

FIGS. 15A-15B illustrate further embodiments having different arrangement of cavities within a carousel;

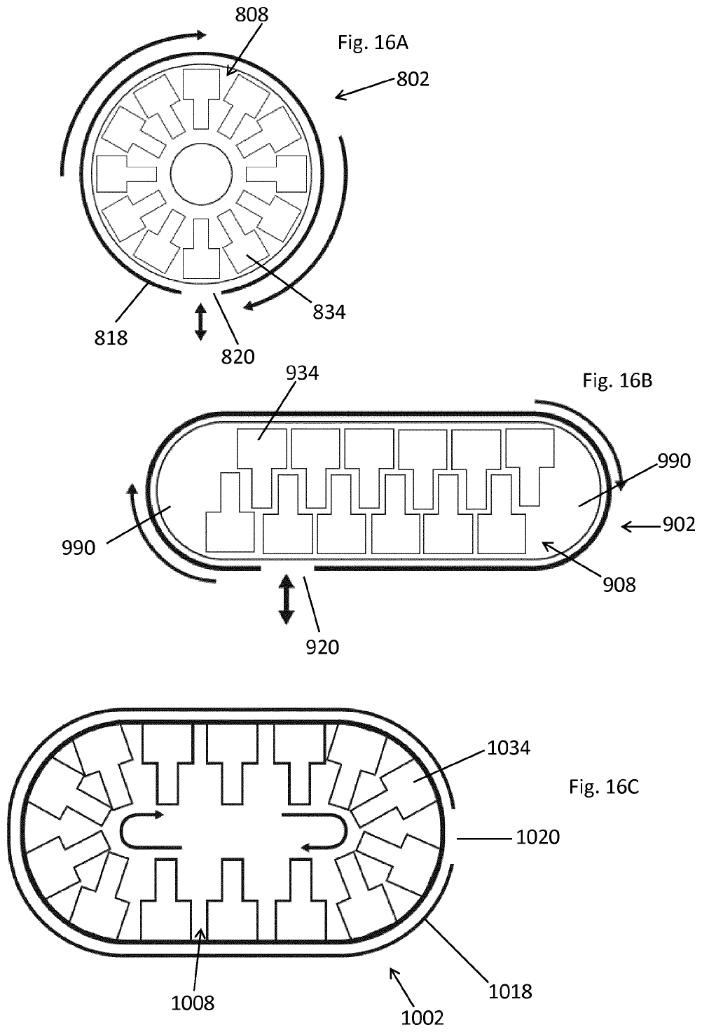

FIGS. 16A-16C illustrate the operation of different storage devices;

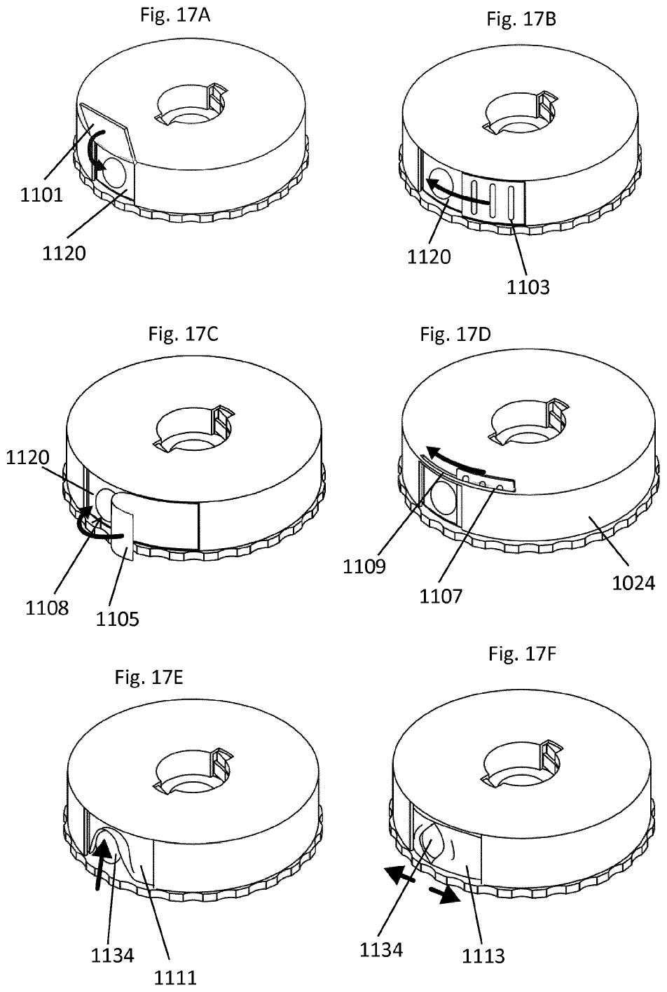

FIGS. 17A-17F illustrate different means for closing an opening on a storage device;

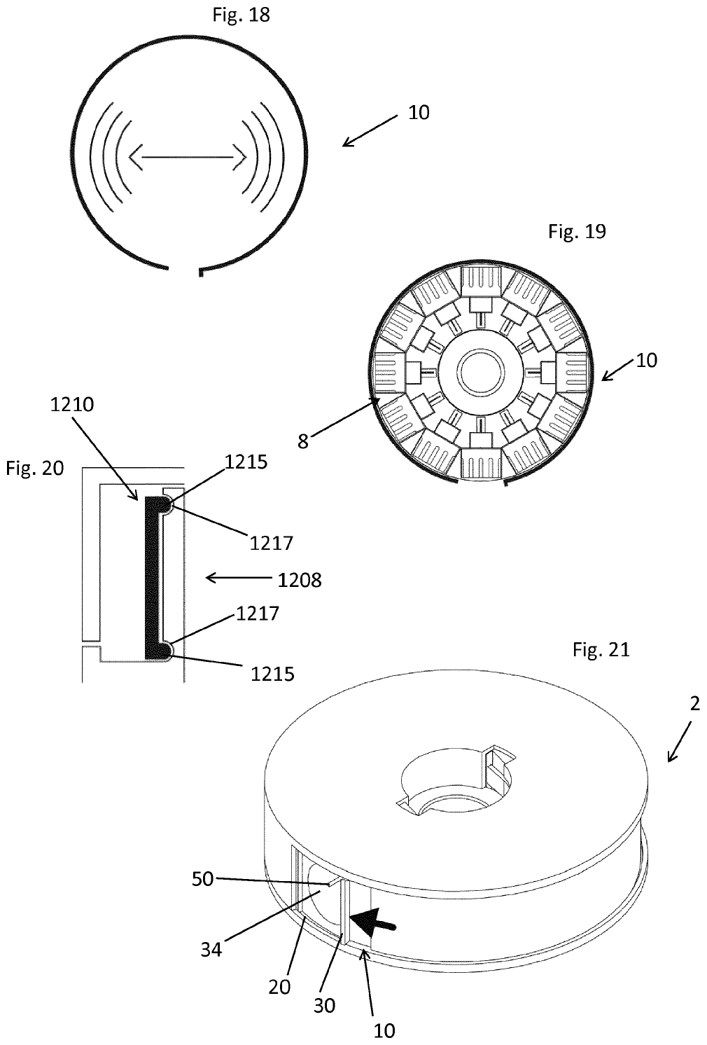

FIG. 18 illustrates a band in accordance with an embodiment of the present invention;

FIG. 19 illustrates the band of FIG. 18 wrapped around a carousel;

FIG. 20 illustrates the interaction between a band and a carousel;

FIG. 21 illustrates a band functioning as a shutter;

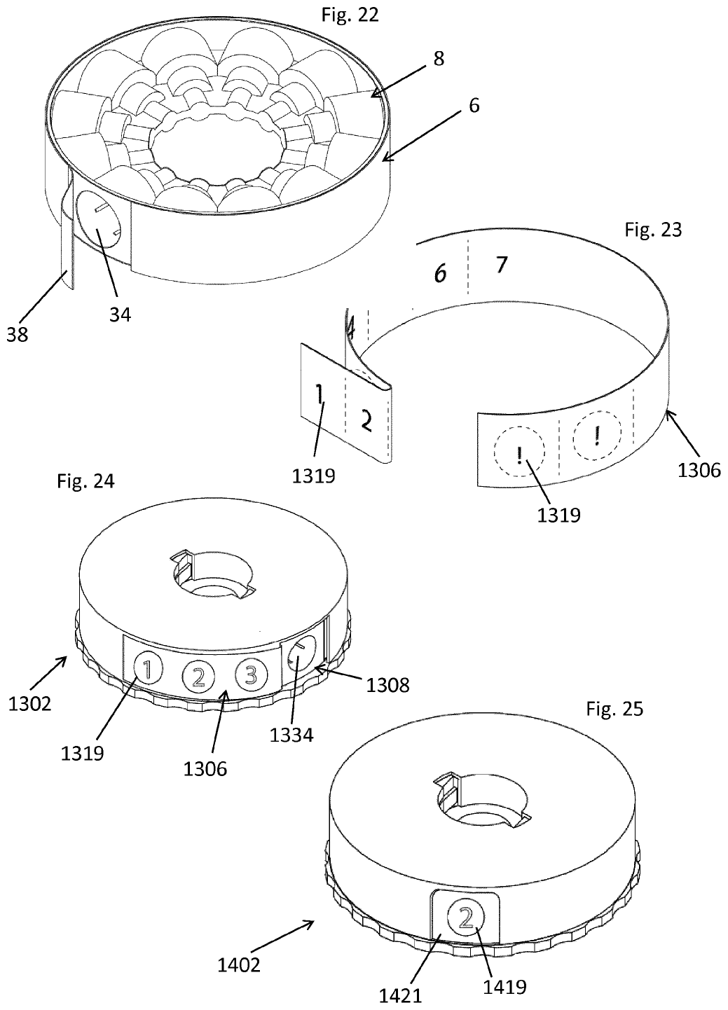

FIG. 22 illustrates a single sealing strip sealing a plurality of cavities on a carousel;

FIG. 23 illustrates a sealing strip which comprises information indicative of the state of the storage device;

FIG. 24 shows a sealing strip as seen in FIG. 23 being pulled around a guide structure to reveal status information;

FIG. 25 illustrates a storage device with a window to view status information;

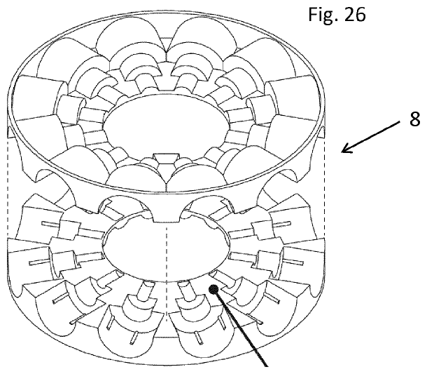

FIG. 26 illustrates a carousel formed from two separate moulded halves;

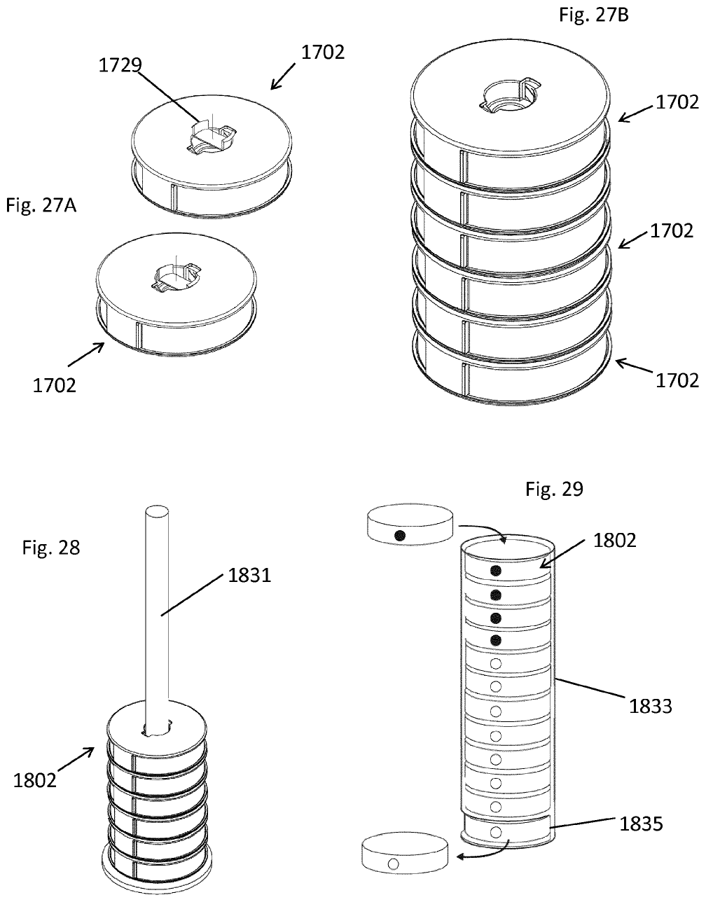

FIGS. 27A-27B illustrate how multiple storage devices may be stacked together;

FIG. 28 illustrates how a plurality of storage devices may be stored on a single pole;

FIG. 29 illustrates how a plurality of storage devices may be contained within a dispensing container; and

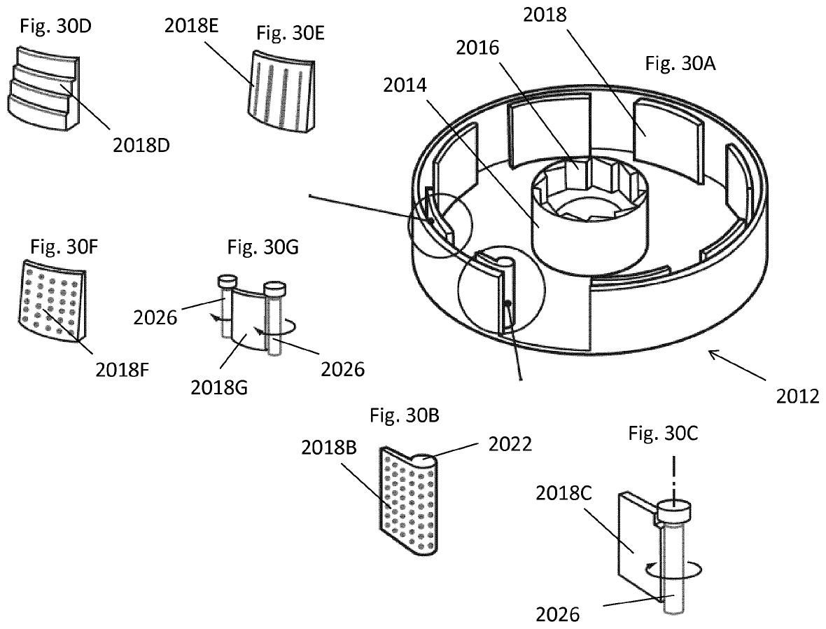

FIGS. 30A-30G illustrate some alternative guide structures in other embodiments of a storage device in accordance with the present invention.

The following Figures focus on a storage device which is designed to store and dispense pen needles for insulin pens. However, as discussed previously, the Applicant has appreciated that the storage device may be designed so as to store a wide variety of different objects and therefore the discussion below which is focussed on storing pen needles is merely an example of one possible use of a storage device in accordance with the present invention.

FIG. 1 illustrates an exploded view of a storage device 2 in accordance with an embodiment of the present invention. In this particular embodiment, the storage device 2 comprises five main components including: a driving cover 4, an elongate sealing strip 6, a carousel 8, a band 10 and a base cover 12. The base cover 12 comprises a hollow cylindrical protrusion 14 extending from its centre. The cylindrical protrusion 14 further comprises geared teeth 16 on its interior which form part of a ratchet mechanism. The base cover 12 further comprises a guide structure formed as a concentric guide wall 18 which extends substantially around the base cover 12 and forms an opening 20. A rounded end 22 is formed on one edge of the guide wall 18. An outer wall 24 extends around the guide wall 18 to a similar extent so as to form a corresponding opening 20. The two concentric walls 18, 24 form a narrow annular slot 26.

The band 10 comprises a main ring section 28 extending for the majority of a circle and a perpendicular protrusion 30. The band 10 is preferably formed from a partially resilient material with a natural radius that is less than the radius of the carousel 8 so that when the band 10 is placed around the carousel 8 it tends to grip it. The carousel 8 is formed in a ring shape with an aperture 32 at its centre. The aperture 32 has an internal diameter which is equal to the external diameter of the cylindrical protrusion 14 on the base cover 12 to allow them to mutually rotate. The carousel 8 seen in this Figure has twelve cavities 34 that are shaped so as to receive pen needles.

The sealing strip 6 comprises an elongate sealing portion 36 and a tab or attachment portion 38. The driving cover 4 comprises a circular shaped disk portion 40, and a driving member in the form of a driving arm 42. The attachment portion 38 of the sealing strip 6 may be attached to the driving arm 42 e.g. by a special adhesive pad (not shown) which has a stronger adhesive than the rest of the strip. The disk portion 40 comprises a cylindrical protrusion 44 extending from its centre which also comprises pawls 46 designed to engage with the geared teeth 16. Additionally, the disk portion 40 is provided with a plurality of gripping features 48 formed as scalloped sections.

The assembly of the main parts of the storage device 2 will be described in relation to FIGS. 3A-3D below.

FIGS. 2A-2G illustrate how the storage device 2, seen in FIG. 1, may be used to dispense insulin needles. FIG. 2A shows the storage device 2 prior to first use, when the twelve cavities 34 are filled with insulin pen needles 50. It can be seen that the elongate sealing portion 36 of the sealing strip 6 passes around the carousel 8 and seals all of the cavities 34. In this initial state the driving arm 42 is located at the opening 20 and acts to close the opening 20. Also, the band 10 is positioned such that the protrusion 30 abuts against an end of the guide wall 18. The band 10 is held in this position by the driving arm 42. The attachment portion 38, of the sealing strip 6, is attached to the driving arm 42, and at this stage where no cavities have been unsealed, the elongate sealing portion 36 does not yet pass around the rounded end 22 of the guide wall 18.

When a user wishes to remove one of the insulin needles 50 from the storage device 2, they must grip the scalloped sections of the driving cover 4 and twist it relative to the base cover 12 (clockwise in this illustration) moving the driving arm 42 around the annular slot 26 so as to pull the elongate sealing portion 36 away from the carousel 8 and unseal a cavity. The point at which a user has unsealed one cavity can be seen in FIG. 2B. The driving arm 42 has been moved to a first position in which the elongate sealing portion 36 has been pulled away from the carousel 8 so as to unseal a cavity 34a. It can be seen that at this first point the elongate sealing portion 36 is now divided into a portion which remains sealed to the carousel 8 and a released portion 52 which doubles back as it begins to be pulled around the guide wall 18 into the annular slot 26 formed between the guide wall 18 and the outer wall 24. Once the driving arm 42 has been moved to this first position a user may access a needle 50a via the opening 20, to take it out of the cavity 34a and, following use, replace it. Once the needle 50a has been used and replaced, a user may then proceed to close the device.

The procedure for closing the device can be seen in FIG. 2C. The user pulls the band 10 around the carousel 8 by applying a force to the protrusion 30. This will cause the band 10 to move around the carousel 8 (which remains static) into a position which closes the opening 20. In this position the carousel 8 is effectively closed off, particularly the opening 20 to the unsealed cavity 34a which contains the used insulin needle 50a. This may be particularly advantageous, in preventing any undesirable smells from coming from the storage device 2.

FIGS. 2D and 2E illustrate the point at which the driving arm 42 is rotated further around the annular slot 26 by twisting the driving cover 4 so as to unseal a second cavity 34b and reveal another unused insulin needle 50b. As the driving arm 42 pulls on the elongate sealing portion 36 it moves around the rounded end 22. The purpose of the rounded end 22 is to facilitate smooth movement of the elongate sealing portion 36, and thereby smooth movement of the other components. As the driving arm 42 pulls the sealing strip around the rounded end 22 it therefore unpeels the strip from the next cavity 34b and in doing so drags the carousel 8 round relative to the base cover 12 in the opposite direction to the driving cover 4 (i.e. anti-clockwise).

Although not explicitly shown in FIGS. 1 and 2, the guide wall 18 may have a low friction surface for reducing the friction between the guide structure and the sealing strip. In some examples, the guide wall 18 and the sealing strip may be produced in materials optimised for low friction. In some examples, in addition or alternatively, a low friction interaction can be provided by the design of structures on the surface of the guide wall 18, in the lateral direction compared to the sealing strip and/or perpendicular to the sealing strip. In some examples, indents or protrusions can be distributed in a regular or random pattern around the surface of the guide wall 18. In some examples, in addition or alternatively, a low friction and smooth operation between the guide wall 18 and the sealing strip can be achieved by adding mechanical aid. Such mechanical aid can be one or more rollers or wheels with or without bearings. For example, the rounded and 22 may comprise a cylindrical roller. The upper part of the roller may rest against the inner carousel 8 for structural support. Such reduced friction and smooth operation is important around the complete guide wall 18 but it is particularly important at the rounded end 22 where the sealing strip is turned through 180°.

As the band 10 has a smaller natural radius than the carousel 8, it positively grips onto the carousel 8. When the elongate sealing strip 36 is pulled away from the carousel 8 it will cause the band 10 to rotate anti-clockwise with it and reveal opening 20 along with the unsealed cavity 34b and sterile needle 50b as shown in FIG. 2E. This operation is particularly elegant in revealing a fresh sterile needle 50b with minimal effort by a user. However if the grip of the band is not tight enough the user may then manually move the band 10 by applying a force to the protrusion 30 in an anti-clockwise direction to move from FIG. 2D to 2E.

The geared teeth 18 and the pawls 46 prevent the driving arm 42 from moving anti-clockwise and thus only allow new, previously unsealed, cavities 34 to be revealed. This prevents a user from accessing already used needles 50.

Once the next needle 50b has been removed and replaced after use, the band 10 can once more be moved across the protrusion 30 to close the opening. FIG. 2F illustrates a similar stage to that seen in FIG. 2C where the band 10 has, once more, been manually moved by the user so as to close the opening 20.

The operational steps described above may be repeated until all of the cavities 34 have been unsealed. It may not be necessary to reclose the opening 20 with the band 10 after each use and further it may not be necessary to replace the insulin needles 50 back into the cavity prior to unsealing the next cavity. In certain circumstances it may be necessary to dispose of the insulin needles 50 via another means.

A user may therefore proceed to unseal the cavities 34 and use the needles 50 until the device ends up in the position seen in FIG. 2G. At this point all of the cavities 34 have been unsealed and the driving arm 42 has moved 360 degrees clockwise back to its original position. As a result of the clockwise motion, as the driving arm 42 moves back to its original position it comes into contact with the protrusion 30 on the band 10 and drives the protrusion 30 such that the band 10 closes the opening 20. The opening 20 is then closed by both the band 10 and the driving arm 42. In this position, the elongate sealing portion 36 has been fully wrapped around the guide wall 18. The interaction between the geared teeth 16 and the pawls 46 prevent the driving arm 42 from moving anti-clockwise and thus once the storage device 2 reaches this stage it cannot easily be reversed. However, there may be certain situations, for example in emergencies where a user has run out of sterile needles, that a user needs to reuse an already used needle. Therefore it may be possible to overcome the interaction between the pawls 46 and the geared teeth 16 through a release tab (not shown) to enable a user to reverse the driving arm 42 and gain access to one of the used needles 50.

Certain components of the storage device 2 may be re-usable, for example the driving cover 4 and the base cover 12. A user may use these reusable components multiple times and just replace the carousel 8, comprising a sealing strip 6 and band 10. In this instance it may be necessary for a user to manually assemble the storage device 2 themselves. Assembly of such a device will be explained with reference to FIGS. 3A-3D.

The user is provided with a pre-assembled insert which consists of the carousel 8, pre-filled with a number of needles and fully sealed with the sealing strip 6 such that only the attachment portion 38 is free. The band 10 is also fitted attached around the carousel 8 and sealing strip 6 as seen in FIG. 3A to form the insert. The band 10 advantageously protects the sealing strip 6 from damage.

FIG. 3B illustrates how the user then takes the insert and attaches it to the driving cover 4. The user attaches the driving arm 42 to the attachment portion 38 of the sealing strip 6 by means of the aforementioned strong adhesive patch. In doing so, the user inserts central boss 44 into the aperture 32 in the carousel as seen in FIG. 3C.

The user must then combine the assembly seen in FIG. 3C with the base cover 12, as shown in FIG. 3D. This is done by guiding the central boss 44 into the centre of the extending cylindrical portion 14 on the base cover 12. This allows the carousel 8 and band 10 to rotate concentrically around the cylindrical portion 14 inside the guide wall 18. When attached together the driving arm 42 will be aligned with the opening 20.

FIG. 4 illustrates a further embodiment of a storage device 102 in accordance with the invention. The storage device 102 contains similar components to the storage device 2 seen in FIG. 1. The main difference between the two devices, is that in this embodiment the storage device 102 comprises a plurality of guide wall portions 118 that extend from the base cover 112 rather than a single continuous wall. The guide wall portions 118 are spaced such that when the driving arm 142 drives the sealing strip around them, the sealing strip does not get caught in the gaps between them.

FIG. 5 illustrates a further embodiment of a storage device 202 in accordance with the present invention. This embodiment also has similar components to the storage device 2 seen in FIG. 1. The main difference is that the guide wall 218 is formed as a physically separate part to the base cover 212. The guide wall 218 comprises a plurality of protrusions 252 and the base cover 212 comprises a plurality of corresponding recesses 254. The protrusions 252 and recesses 254 are shaped to engage with one another to hold the base cover 212 and guide wall 218 together once assembled. This embodiment may be advantageous as it may be easier to manufacture. The guide wall 218 further comprises a plurality of circular cut-outs 256 extending around the guide wall 218. The cut-outs 256 reduce the total amount of material required to make the guide wall 218 without reducing its ability to guide the sealing strip 206.

FIG. 6 illustrates a further embodiment of a storage device 302 in accordance with the present invention. This too is similar to the storage device 2 seen in FIG. 1 except that a band 310 and a small section of guide 358 with rounded edge 322 together form a guide structure instead of a guide wall. The band 10 has a similar shape to the band 310 seen in FIG. 1, however it further comprises a series of ribs 360 which are spaced around the circumference of the band 310 and extend along the width of the band 310. During use, the sealing strip 306 is pulled by the driving arm 342 around the guide 358 and then around the band 310, passing around each of the ribs 360 which prevent too much friction building up between the strip 306 and the band 310.

FIG. 7 illustrates a further embodiment of a storage device 402 in accordance with the present invention. Unlike the previous embodiments, the storage device 402 does not comprise a base cover. Instead, the carousel 408 forms the base of the storage device 402. As a result of not having a base cover, there is not a guide wall provided in a base cover. Instead, a guide structure is provided by a guide band 418. The guide band 418 functions to guide the sealing strip 406 as the elongate sealing portion 436 is pulled away from the cavities 434. The guide band 418 is very similar in appearance to the band in previous embodiments which grips the carousel. In order to retain the guide band 418 when around the carousel 408, the carousel 408 comprises a circumferential rim 462 which extends around it. When the guide band 418 is placed around the carousel 408 it is held in position by the rim 462. The guide band is prevented from moving upward and away from the carousel 408 by the driving cover 404. The driving cover 404 comprises a similar disk portion 440 and a driving arm 442 to that seen in previous embodiments. The driving cover also comprises a central boss 44 and pawls 46. The central boss 44 fits into a central aperture 432 in the carousel 408. Unlike the previous embodiments, the aperture 432 in the carousel 408 comprises geared teeth 416 to engage the pawls 446. When assembled, the driving arm 442 sits on the outside of the guide band 418 and when the driving arm 442 is moved relative to the carousel 408 it drives the sealing strip 406 around the guide band 418.

As with previous embodiments, the carousel 408 comprises a plurality of cavities 434 capable of housing insulin needles (not shown). It can also be seen that the guide band 418 comprises a protrusion 430.

Operation of the storage device 402 seen in FIG. 7 will now be described with reference to FIGS. 8A-8D. FIG. 8A illustrates the storage device 402 when the plurality of cavities 434 contain insulin needles 450 and are all sealed by the sealing strip 406. At this stage, the driving arm 442 abuts against the protrusion 430. FIG. 8B illustrates the storage device 402 after a first cavity 434a has been unsealed to allow the first needle 450a to be removed. It can be seen that the driving arm 442 has been driven in a clockwise direction and has pulled the sealing strip 406 around the guide band 418. When considering the relative movement of the components, the carousel 408 may be considered to remain stationary, and the driving arm 442 may be considered to move around the carousel 408 along with the guide band 418. The movement of the driving arm 442 and the guide band 418 allows access to the different cavities 434a, b etc. Similarly to previous embodiments, the driving arm 442 wraps the sealing strip 406 around the guide band 418.

However, unlike previous embodiments, the storage device 402 does not have any means for closing the cavity 434 which is present at the opening of the guide band 418 when a user has replaced a used needle. Therefore, the next stage in the process when a user next wants to remove another needle 450b from a cavity is simply to move the driving arm 442 further around the carousel 408, peeling more of the sealing strip 406 away from the carousel 408 thus revealing a further cavity 434b so that the needle 450b can be removed as shown in FIG. 8C.

This process may be repeated until the storage device 402 reaches the configuration seen in FIG. 8D. Here the driving arm 442 has been rotated 360 degrees around the carousel 408 and pulled the sealing strip 406 such that all of the cavities 434 have been unsealed. At this point the driving arm 442 comes into contact with the protrusion 430 on the guide band 418 and the driving arm 434 cannot be moved any further. Backwards movement, i.e. anticlockwise movement, is prevented by engagement of the geared teeth 416 and the pawls 446. Engagement between these two features also prevents anticlockwise movement of the driving arm 434 throughout operation of the storage device 402.

FIG. 9 illustrates an embodiment of a storage device 402 in accordance with another aspect of the present invention. The storage device comprises a cover 504, a looped sealing strip 506 and a carousel 508. Unlike previous embodiments where the guide structure is provided on the base cover or as a separate band, in this embodiment the guide structure is provided in the cover 504 as a guide wall 518. Whilst not explicitly shown, the guide wall 518 may take a similar form to the guide wall seen in FIG. 1. The sealing strip 506 comprises a first portion 564 and a second portion 566 which are joined to form a continuous loop. Initially the first portion 564 is sealed to the carousel 508 and the second portion 566 passes around the guide wall 518. Due to the opening 568 in the cover 502, and the relative position of the guide wall 518, in order for the sealing strip 506 to be sealed to the carousel 508 and pass around the guide wall 518 with sufficient tension such that no bunching of the sealing strip 506 occurs one of the cavities 534 on the carousel 508 is not sealed by the sealing strip. Therefore, to seal this cavity 534, a separate individual seal 570 with a sealing portion 572 and a tab 574 is provided.

The carousel 508 comprises geared teeth 516 which engage with the pawls 546, located on the cover 504, to ensure that the carousel 508 can only move in one direction with respect to the cover 504 and therefore only reveal sterile, previously sealed, cavities. The carousel 508 comprises a circumferential lipped section 578 which accommodates the cover 504 and the guide wall 518 when the cover 504 is placed onto the carousel 508. The lipped section 578 further comprises a plurality of scalloped sections 580 around its circumference. In use a user may grip the carousel 508 and rotate the cover 504. The scalloped sections 580 improve the user's grip on the device 502. Whilst not shown, the cover 504 may also comprise gripping features to assist the user when operating the device 502.

It will be appreciated that relative movement between the carousel 508 and the guide structure 518 is caused by the geared teeth 516 engaging with the pawls 546. As is mentioned above, in use a user may grip the carousel 508 and rotate the cover 504 so as to rotate the guide wall 518 relative to the carousel. The cover 504 is therefore acting as the driving member, i.e. a driving cover 504. Alternatively, in use a user may grip the cover 504 and rotate the carousel 508 so as to cause relative movement between the carousel 508 and the guide wall 518 extending from the cover 504. The carousel 508 is therefore acting as the driving member. Both the cover 504 and carousel 508 comprise a planar member that extends in a plane substantially parallel to the plane of the carousel 508.

Operation of the storage device 502 will now be described with reference to FIGS. 10A and 10B. FIG. 10A illustrates the device 502 prior to use, when all of the cavities 534 are sealed. It can be seen that the individual seal 570 may be removed to reveal the sterile needle 550 in the cavity 534. Once the needle 550 has been used and, if desired, replaced, the storage device 502 can then be operated to peel away the sealing strip 506 from the remaining cavities 534 to allow access to other needles 550.

FIG. 10B illustrates the device 502 at a point at which the carousel 508 has been rotated relative to the cover 504 such that the sealing strip 506 has been peeled away from another cavity 534b. It will be appreciated that as soon as a portion of the sealing strip is peeled away from the carousel 508, a corresponding portion of the sealing strip which was previously around the guide wall 518 is brought back onto the carousel 508. Whilst it may no longer actually fully seal against the carousel 508, it may press against the carousel 508 with sufficient force such that it at least partially reseals the cavities 534. This process of peeling a part of the sealing strip 506 away and then adding a corresponding portion back on to the carousel 508 to the opposite side of the carousel continues until all of the sealing strip 506 has been peeled away from the carousel 508. Whilst not shown, there may be an arrangement between the cover 504, sealing strip 506 and cover 508 that prevents the carousel from being rotated more than 360 degrees.

FIG. 11 provides an illustrative example of how the sealing strip 506, seen in the embodiment shown in FIG. 9, passes around the guide wall 518 and carousel 508. As the sealing strip 506 is a single continuous loop, movement of a first portion directly corresponds to movement of a second portion. It can be seen that the loop of the sealing strip 506 extends concentrically around the carousel 508.

FIG. 12 illustrates a storage device 602 which comprises similar components to the storage device 2 seen in FIG. 1. The main difference between the two devices is that the storage device 602 further comprises a carousel ratchet 676 located on the base cover 612. The carousel ratchet 676 engages with a protrusion 678 (not shown) on the carousel 608 and prevents the carousel from moving in one rotational direction. Whilst there is only a single carousel ratchet 676 shown in this Figure, it will be appreciated that a plurality of carousel ratchets could be provided. FIG. 13 shows a cross section of the storage device 602 seen in FIG. 12 and illustrates the interaction between the carousel ratchet 676 and the protrusion 678. The carousel ratchet 676 prevents reverse motion and only allows the carousel 608 to move in one direction. As discussed above, in certain circumstances it may be necessary for a user to access an already unsealed cavity, in such circumstances a user may be able to manually overcome the carousel ratchet 676 so as to permit reverse motion of the carousel 608. This may be achieved by applying a sufficiently large force to the carousel ratchet 676 causing it to deform and move out of the way of the protrusion 678. Alternatively it may be that the carousel ratchet 676 is actually caused to break away to permit reverse motion.

FIGS. 14A-14D illustrate some examples of different carousel shapes which may be employed in accordance with the invention. FIG. 14A illustrates a carousel 780 in the form of an oblong with rounded ends. The cavities 734 feature around the entire perimeter of the carousel 780. FIG. 14B illustrates a different carousel 784 in the form of a triangle with rounded vertices. Similarly to the carousel 780, the cavities 734 feature around the entire perimeter 786 of the carousel 784 and all point generally in the direction of the centre of the carousel 784.

FIG. 14C shows another alternative carousel 788 in the form of a narrow oblong. Here it can be seen that the cavities 734 are tessellated within the carousel 788 and as a result of the narrow shape, there are no cavities present at the rounded ends 790.

It will be appreciated that the carousel may take many different forms. The guide structure and driving member will have to be shaped and arranged to work in accordance with different shaped carousels so as to pull the sealing tape away from the carousel.

FIG. 15A illustrates a carousel 792 which has a singular layer of cavities 734 of the type shown in earlier embodiments. FIG. 15B illustrates an alternative carousel 794 which comprises a first layer of cavities 734 and a second layer of cavities 796. The second layer of cavities may be positioned such that they are aligned with the wall sections 798 which separate the cavities on the first layer of cavities 734 i.e. the two layers can be interleaved. Alternatively they may be positioned directly above the first layer of cavities 734 such that two cavities 734, 796 are presented at any one time. It will be appreciated that the device would require an opening shaped so as to reveal both cavities 734, 796 in this instance. Revealing two cavities at the same time may be useful when it is required to dispense two objects at the same time which would be unstable if stored together. For example, there are certain pharmaceuticals which cannot be stored together but are administered together.

FIGS. 16A and 16B show schematic diagrams of the operation of a storage device in which an outer wall with an opening, which may be a guide wall or simply an outer wall comprising an opening, is moved around the carousel to reveal each cavity. FIG. 16A shows a storage device 802, wherein the carousel 808 remains in a fixed position and the guide wall 818 moves around the carousel 808 to move the opening 820 relative to the cavities. FIG. 16B shows a similar operation but with a carousel 908 which has a different shape. In this embodiment, when the opening 920 is moved along the carousel 908, when it reaches one end portion 990, it will have to be passed around the end portion 990 before a further cavity 934 is reached.

FIG. 16C shows an alternative embodiment in which the guide wall 1018 an opening 1020 remain in a fixed position and the carousel 1008 is formed as a belt which moves within the guide wall 1018 so as to present a cavity 1034 at the opening 1020. It will be appreciated that due to the non-circular shape of the device, the carousel 1008 must be formed as a flexible belt to allow it to move within the guide wall 1018. Whilst not shown in FIGS. 16A-16C, in practice there would be a sealing strip along with a driving member arranged to pull the sealing strip around said guide wall.

As discussed previously, there may be many different ways of closing the opening formed on an outer wall and/or guide structure on the device. FIGS. 17A-17F illustrate various different examples of how the opening may be closed. FIG. 17A shows a storage device which comprises a shutter in the form of a hinged door 1101 which is arranged to close the opening 1120.

FIG. 17B illustrates an alternative embodiment wherein the shutter is in the form of a sliding door 1103 which slides around the circumference of the storage device to open and close the opening 1120.

FIG. 17C illustrates an embodiment of the storage device wherein a shutter is provided by an adhesive strip 1105 which may adhere to the carousel 1108 or to the outer surface of the storage device 1102.

FIG. 17D illustrates a storage device which comprises a sliding door 1107 which is similar to that of FIG. 17B except that it is contained within an outer wall 1024 of the storage device 2. This sliding door 1107 may be an independent door or alternatively it may be part of the band seen in previous embodiments. As seen in this Figure the sliding door may protrude through a slot 1109 on the top of the storage device.

FIG. 17E illustrate a further embodiment of a storage device wherein a shutter is provided by a flexible silicon seal 1111 which may be pushed upwards to reveal a cavity.

FIG. 17F illustrates a further alternative embodiment, wherein a different silicon seal 1113 is provided which allows the user to push through the seal 1113 to gain access to the cavity 1134. The Applicant has recognised that these are only a small number of examples of how a shutter may be provided.

FIG. 18 shows the band 10, seen in FIG. 1, viewed from above. This Figure demonstrates how the band 10 may be formed from a resilient material, for example plastic, such that when it is deformed it acts to return to its original shape. As seen in FIG. 19, when the band 10 is placed around the carousel 8, as it has a smaller natural radius than the carousel 8, it is expanded so as to fit around the carousel. The result is that the band 10 positively grips onto the carousel 8.

The resiliency of the material from which the band is manufactured may be sufficient that the band 10 grips onto the carousel 8 and cannot easily become free. However, the Applicant has recognised that the resilience of the material alone may not sufficient to ensure that the band 10 is retained on the carousel. Therefore, in FIG. 20, there is shown a further embodiment wherein the band 1210 comprises a rim 1215 at its top and bottom that is shaped so as to engage with a slot 1217 on the carousel 1208. This may help to locate and hold the band 1210 on the carousel 1208. As discussed previously the band 1210 may form the guide structure and act to reseal unsealed cavities, or alternatively it may solely be for the purpose of resealing unsealed cavities. This particular arrangement whereby the band 1206 comprises rims 1217 which engage with slots 1217 on the carousel 1208 may be particularly well suited to resealing the carousel 1208 as it will likely provide a more air-tight seal between individual cavities.

FIG. 21 shows an assembled view of the storage device 2 seen in FIG. 1. This Figure also illustrates how the section of the band 6 which comprises the protrusion 30 can be located within the opening 20. It is possible to slide the band 6 so as to close the opening and close the needle 50 within the cavity 34.

FIG. 22 shows a sealing strip 6 sealed around the carousel 8. It can be seen that the single sealing strip 6 acts to seal a plurality of the cavities 34 on the carousel and therefore individual seals are not required.

FIG. 23 illustrates a sealing strip which comprises status information 1319 in the form of numbers or warning symbols. The status information 1319 may be printed on either the inside surface of the sealing strip 1306 or on the outside surface depending on how the particular storage device which it is intended to be used with works.

FIG. 24 shows a storage device 1302 comprising a sealing strip 1306 which has status information 1319 printed thereon. It can be seen that as the sealing strip 1306 is peeled away from the carousel, it is driven around the outside of the storage device 1302 and so the information 1319 is visible to the user. In this case the information 1319 will be printed on the inside surface of the sealing strip 1306, i.e. the surface which is sealed against the carousel. In this instance the information 1319 indicates to the user how many cavities 1334 have been unsealed.

FIG. 25 illustrates a further embodiment of a storage device 1402 which provides status information to a user. In this embodiment status information 1419 is visible through a window 1421 on an outer wall of the storage device 1402. The window may be in the form of an opening or alternatively it may comprise a transparent or translucent material to allow the user to see the status information 1419.

In the examples shown, the cavities within the carousels are shaped so as to contain insulin needles. Due to the relatively complicated shape required to contain insulin needles the carousel may be difficult to manufacture. FIG. 26 illustrates a method of producing a carousel with cavities with a complicated shape. The carousel 8 may be formed from two separate halves which may be welded together or attached by any other suitable means. Using this technique it may be possible to easily produce cavities with a relatively complicated shape.

The storage device or components thereof may be disposable. As a result a number of disposable storage devices, or components, may be provided to a user. The storage devices may be provided in the form of a stacked structure. This is illustrated in FIGS. 27A and 27B. In FIG. 27A it can be seen that the storage devices 1702 comprise stacking tabs 1729 on the top surface of the storage device 1702 which engage with an underside of a second storage device 1702. By using the stacking tabs 1729 it may be possible to stack a plurality of storage devices 1702 together to form a stacked structure as seen in FIG. 27B.

Alternatively, in embodiments where the storage device comprises an aperture, the storage devices may be stacked onto a pole 1831 as seen in FIG. 28. As a further alternative, storage devices 1802 may be stacked in a container 1833 with an opening 1835 at the bottom allowing a user to extract a storage device 1802 from the container 1833. Once a storage device has been used, the used storage device may be replaced into the top of container 1833. This can be seen in FIG. 29.

FIGS. 30A-30G illustrate some alternative guide structures that may replace the guide wall 18 seen in FIG. 1, or the guide wall portions 118 seen in FIG. 4, or may be combined with any of the other embodiments described above as appropriate. There is seen in FIG. 30A a base cover 2012 comprising a hollow cylindrical protrusion 2014 extending from its centre and carrying geared teeth 2016 to form part of a ratchet mechanism to drive relative movement between the base cover 2012 and a driving member (not shown) attached to a sealing strip (not shown). In these embodiments the guide structure comprises a plurality of discrete guide wall portions 2018 that extend axially upwards from the base cover 2012. As will be appreciated form the description above, in a complete device a sealing strip such as the sealing strip 6 seen in FIGS. 1 and 4 will wrap around the outside of the guide wall portions 2018.

In the embodiment seen in FIG. 30B, the guide wall portion 2018B comprises a rounded end 2022 provided with a low friction surface 2024. In the embodiment seen in FIG. 30C, the guide wall portion 2018C comprises a roller 2026 at one end. In both cases there is a reduction in the friction experienced by a sealing strip as it is pulled around the corner at the end of the guide wall portion 2018.

In the embodiment seen in FIG. 30D, the guide wall portion 2018D comprises a stepped surface. In the embodiment seen in FIG. 30E, the guide wall portion 2018E comprises a ridged surface. In the embodiment seen in FIG. 30F, the guide wall portion 2018F comprises a dimpled or knobbly surface. In the embodiment seen in FIG. 30G, the guide wall portion 2018G comprises rollers 2026 at both ends.

One or more of the guide wall portions 2018B-2018G may be arranged alone or in combination to form the guide structure in any given device.

1. A storage device for storing a plurality of objects, comprising:
a carousel defining a plurality of cavities;
a sealing strip extending around the carousel so as to seal the plurality of cavities;
a concentric guide structure extending at least partially around the carousel; and
a driving member attached to the sealing strip and arranged, when moved relative to the guide structure, to pull on the sealing strip and thereby release a portion of the sealing strip and subsequently to wrap said portion around the guide structure.

2. A storage device for storing a plurality of objects, comprising:
a carousel defining a plurality of cavities;
a concentric guide structure extending around the carousel; wherein the guide structure comprises an opening to allow access to at least one of the plurality of cavities; and
a sealing strip formed as a loop, wherein a first portion of the loop extends around the carousel to seal the plurality of cavities and a second portion of the loop extends, via the opening, around the guide structure.

3. The storage device as claimed in claim 2, wherein the storage device further comprises a driving member arranged to cause relative movement between the carousel and the guide structure, and optionally wherein the driving member comprises a planar member that extends in a plane substantially parallel to a plane of the carousel.

4. (canceled)

5. The storage device as claimed in claim 1, wherein the driving member comprises a planar member that extends in a plane substantially parallel to a plane of the carousel.

6. The storage device as claimed in claim 5, wherein the driving member comprises an extending portion which extends in an axial direction with respect to the carousel and is arranged to engage the sealing strip, and optionally wherein the extending portion extends from an edge of the planar member.

7. (canceled)

8. The storage device as claimed in claim 6, wherein the guide structure follows a circular arc with a first radius and wherein the extending portion follows a circular arc with a second radius, wherein the second radius is larger than the first radius.

9. The storage device as claimed in claim 1, wherein the driving member comprises an external surface including a plurality of gripping features which may be engaged by a user.

10. (canceled)

11. (canceled)

12. The storage device as claimed in claim 1, comprising a ratchet arranged to restrict relative movement between the driving member and the guide structure.

13.-18. (canceled)

19. The storage device as claimed in claim 1, comprising a base cover for the storage device and the guide structure being formed as an integral part of the base cover.

20.-24. (canceled)

25. The storage device as claimed in claim 1, wherein the guide structure is formed from a single, continuous, guide wall that extends at least partially around the carousel.

26. (canceled)

27. (canceled)

28. The storage device as claimed in claim 25, wherein the guide structure extends at least 270° around the carousel.

29. The storage device as claimed in claim 1, wherein the guide structure comprises at least one opening.

30. The storage device as claimed in claim 29, wherein the at least one opening has a suitable dimension to fully expose no more than one cavity of the plurality of cavities at any one time.

31. (canceled)

32. (canceled)

33. The storage device as claimed in claim 1, wherein the carousel has a circular structure, and wherein each of the plurality of cavities extends radially away from a center of the carousel.

34.-36. (canceled)

37. The storage device as claimed in claim 1, comprising a band that at least partially surrounds the carousel, wherein the band has a natural radius which is smaller than the radius of the carousel and is at least partially resilient so as to grip onto the carousel when the band is placed around the carousel.

38. (canceled)

39. (canceled)

40. The storage device as claimed in claim 29, comprising a band that at least partially surrounds the carousel, the band comprising a protrusion extending into the at least one opening to provide a moveable shutter arranged to close the at least one opening.

41.-54. (canceled)

55. The storage device as claimed in claim 1, wherein the storage device stores a plurality of pen needles and all of the pen needles in their respective cavities using a single sealing strip.

56.-61. (canceled)

62. The storage device as claimed in claim 2, wherein the loop extends concentrically around the carousel.

63. (canceled)

64. The storage device as claimed in claim 1, wherein the sealing strip when released is stored concentrically around the carousel.

65. The storage device as claimed in claim 1, wherein the driving member is arranged to move concentrically around the carousel.

66.-71. (canceled)
